SEQUENCE LISTING (15 sequences)

SEQ ID NO: 1 - DNA, 12 bp, synthetic construct
  source 1..12: mol_type = other DNA
cgtctcacaccg

SEQ ID NO: 2 - DNA, 11 bp, synthetic construct
  source 1..11: mol_type = other DNA
gtttcgagacg

SEQ ID NO: 3 - DNA, 20 bp, synthetic construct
  source 1..20: mol_type = other DNA
aggcacttgctcgtacgacg

SEQ ID NO: 4 - DNA, 20 bp, synthetic construct
  source 1..20: mol_type = other DNA
atgtgggcccggcaccttaa

SEQ ID NO: 5 - DNA, 23 bp, synthetic construct
  source 1..23: mol_type = other DNA
agggcacccggttcatacgcngg

SEQ ID NO: 6 - DNA, 20 bp, synthetic construct
  source 1..20: mol_type = other DNA
gggaacgagcgcgatcacca

SEQ ID NO: 7 - DNA, 20 bp, synthetic construct
  source 1..20: mol_type = other DNA
ggtagggccctggacccgcg

SEQ ID NO: 8 - DNA, 20 bp, synthetic construct
  source 1..20: mol_type = other DNA
cgcgccacagttgggaaccg

SEQ ID NO: 9 - DNA, 20 bp, synthetic construct
  source 1..20: mol_type = other DNA
ccagggacttggaggtggag

SEQ ID NO: 10 - DNA, 131 bp, Homo sapiens
  source 1..131: mol_type = genomic DNA
  misc_feature 65..131: note = coding sequence
acgtttcagagaggctgcagcccggcgcagcatcctgagcgcgcctctgccgaggcgagcggacatgatgcaggctccccgcgcagccctagtcttcgccctggtgatcgcgctcgttcccgtcggccggg

SEQ ID NO: 11 - DNA, 31 bp, Homo sapiens
  source 1..31: mol_type = genomic DNA
cctgggatcgcgctcgttcccgtcggccggg

SEQ ID NO: 12 - DNA, 76 bp, Homo sapiens
  source 1..76: mol_type = genomic DNA
acgtttcagagaggctgcagcccggcgcagcatcctgagcgcgccctagtcttcgccctcgttcccgtcggccggg

SEQ ID NO: 13 - protein, 17 aa, synthetic construct
GGGGSGGGGSGGGGSGS

SEQ ID NO: 14 - protein, 20 aa, synthetic construct
GGGGSGGGGSGYPYDVPDYA

SEQ ID NO: 15 - protein, 14 aa, synthetic construct
GGGGSGGGGSGGGG

(19) United States
(12) Patent Application Publication
(10) Pub. No.: US 2025/0145926 A1
(43) Pub. Date: May 8, 2025

(54) DEVICES, METHODS, AND SYSTEMS FOR ASSESSING CELL MIGRATION AND RELATED PROCESSES

(71) Applicant: University of Washington, Seattle, WA (US)

(72) Inventors: Nathan Belliveau, Seattle, WA (US); Julie Theriot, Seattle, WA (US); Matthew Footer, Seattle, WA (US)

(73) Assignee: University of Washington, Seattle, WA (US)

(21) Appl. No.: 18/933,845

(22) Filed: Oct. 31, 2024

Related U.S. Application Data
(60) Provisional application No. 63/547,320, filed on Nov. 3, 2023.

(51) Int. Cl. C12M 1/00; C12M 1/12; C12M 1/42

(52) U.S. Cl. CPC C12M 23/34; C12M 25/02; C12M 35/06; C12M 47/04

(57) ABSTRACT

Various implementations described herein relate to devices, systems, and methods for inducing and/or causing cellular migration and uses thereof. Implementations described herein can be used to cause a portion of a cell population to migrate in response to a differential stimulus. Implementations described herein can be used to isolate and characterize the portion of the cell population. Implementations described herein can be used for research, diagnostic, or therapeutic purposes.

### CROSS-REFERENCE TO RELATED APPLICATION(S)

This application claims the priority of U.S. Provisional Patent Application No. 63/547,320, filed on Nov. 3, 2023, which is incorporated by reference herein in its entirety.

### STATEMENT REGARDING FEDERALLY SPONSORED RESEARCH OR DEVELOPMENT

This invention was made with government support under Grant No. 1 K99GM147355-01, awarded by the National Institute of General Medical Sciences (NIGMS) [NIH]. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is W149-0059US-Seq.xml. The file is 14,565 bytes, was created Oct. 31, 2024, and is being submitted electronically via Patent Center.

### TECHNICAL FIELD

This application relates to systems and methods for inducing and/or characterizing cell migration. It relates more specifically to causing and/or assessing cell migration in response to a stimulus, such as a chemical stimulus or an electric field.

### BACKGROUND

Directed cell migration is important for many aspects of biology. In mammals, directed migration drives processes critical to development (Barriga and Theveneau, Frontiers in Physiology 11 (2020)), immune system function (T. Lämmermann, et al., Nature 498, 371-375 (2013)), and tissue regeneration after injury (M. Rodrigues, et al., Physiological Reviews 99, 665-706 (2019)). Much of our understanding of how migrating cells effectively sense and respond to environmental signals comes from work on chemotaxis, mediated by transmembrane receptors that bind specific chemical ligands and transduce those signals to reorient the mechanical force-generating components of the cytoskeleton, and thereby direct persistent directional migration (S. SenGupta, et al., Nat Rev Mol Cell Biol 22, 529-547 (2021); Lauffenburger and Horwitz, Cell 84, 359-369 (1996)). Less is understood about how cells sense and integrate other available spatial information about their physicochemical environment, such as gradients in temperature, pH, or matrix stiffness (Shellard and Mayor, Trends in Cell Biology 30, 852-868 (2020)). Directed cell migration in response to acute injury presents a particularly interesting challenge, as the injury must generate a new kind of spatial information directing cells to move toward the wound that was not present in the pre-existing normal structure of the tissue. Here, wound-induced electric fields represent one such directional signal. Nearly all polarized animal epithelia maintain distinct ionic environments on their apical versus basolateral sides due to asymmetric ion transport, and the epithelial barrier presents electrical resistance, normally resulting in a transepithelial potential difference (A. M. Marchiando, et al., Annual Review of Pathology: Mechanisms of Disease 5, 119-144 (2010); T. B. Saw, et al., Nat. Phys. 18, 1122-1128 (2022)). Acute disruption of epithelial barrier integrity by physical injury causes a short-circuit in the transepithelial potential, generating a wound-induced endogenous electric field with magnitudes of 50-500 mV/mm that can persist for many hours (C. E. Pullar, Tissue Regeneration and Cancer, (CRC Press, 2011); B. Reid, et al., The FASEB Journal 19, 379-386 (2005); R. Nuccitelli, et al., Wound Repair and Regeneration 19, 645-655 (2011)). Many cell types, including epithelial cells and tissue-resident immune cells, have been found to migrate directionally in response to such electrical cues in a process called galvanotaxis (or electrotaxis) (M. Zhao, et al., Nature 442, 457-460 (2006)), which has been proposed to play an integral role in the wound healing response (M. Zhao, et al., Cold Spring Harb Perspect Biol 14, (2022)).

Although it has been known for well over a century that many motile cell types can perform galvanotaxis, the molecular mechanism of electric field detection remains elusive. To mediate long-distance directed cell migration, a directional signal generated at the cell surface can be transduced to the mechanical elements of the cytoskeleton. Indeed, several intracellular signaling components known to be involved in chemotaxis have also been shown to contribute to galvanotaxis (Nuccitelli, supra; M. Hu, et al., Sci Rep 14, 3167 (2024); R. Gao, et al., Science Signaling 8, (2015)), indicating that galvanotaxis is a cell-regulated biological response rather than a purely physical response to forces generated by the influence of electric fields on charged macromolecules. However, due to electrostatic shielding and poor conductance across the plasma membrane, it is unlikely that the relatively weak wound-induced electric fields can directly affect the localization or activity of intracellular signaling components (N. M. Belliveau, et al., Nat Commun 14, 5770 (2023); G. M. Allen, et al., Current Biology 23, 560-568 (2013); C. Reily, et al., Nat Rev Nephrol 15, 346-366 (2019); D. L. Bodor, et al., Developmental Cell 52, 550-562 (2020)). While several different kinds of cell surface proteins have been implicated in galvanotaxis, including a voltage-sensitive phosphatase (Gao, supra), the EGF receptor (Petri and Sanz, Cell and Tissue Research 371, 425-436 (2018)), and several ion channels (S. J. Collins, Blood 70, 1233-1244 (1987)), it is not clear whether any of these candidates act as direct sensors of the electric field. For very rapidly moving cells, biophysical experiments and theoretical modeling have most strongly supported a hypothesis that cells can sense the presence and orientation of electric fields via spatial redistribution of charged cell surface macromolecules due to electrophoresis and/or electroosmosis in the plane of the plasma membrane (Belliveau, supra; T. L. Nagy, et al., eLife 12 (2024); Pollard and Borisy, Cell 112, 453-465 (2003)).

### BRIEF DESCRIPTION OF THE DRAWINGS

FIG. 1 illustrates an example environment for causing and/or assessing cell migration.

FIG. 2 illustrates an example environment for causing and/or assessing cell migration.

FIG. 3 illustrates an example process for causing and/or assessing cell migration.

FIGS. 4A-4D illustrate validation of Galvanin knockout cell line and compass autocorrelation analysis. FIGS. 4A and 4B illustrate additional validation to confirm Galvanin gene disruption. FIG. 4A illustrates a western blot confirming loss of Galvanin protein expression. FIG. 4B illustrates histograms summarize directed speed along the electric field vector during migration in a collagen gel. FIG. 4C illustrates a schematic showing calculation of cosine θ, where θ is the angle between the cell trajectory and the electric field vector. FIG. 4D illustrates compass autocorrelation comparing the wild-type HL-60 neutrophils, −/− Galvanin knockout lines, and the genetic rescue (Galvanin-GFP expressed in −/− Galvanin knockout clone 1).

FIGS. 5A-5G illustrate that localization of Galvanin coincides with reduced local membrane speed and front-rear polarization. FIG. 5A illustrates an example analysis showing the temporal movement, Galvanin-GFP fluorescence intensity, and cellular protrusion/retraction activity of a single cell. FIG. 5B illustrates a kymograph showing the normalized Galvanin-GFP intensity, quantified at the cell periphery. FIG. 5C illustrates a kymograph quantifying membrane speed along the cell periphery. FIG. 5D illustrates averaged fluorescence at the anodal side (positions +120 to +240) and cathodal side (positions −60 to +60) during the first 10 minutes. FIG. 5E illustrates averaged membrane speed at the anodal side (positions +120 to +240) and cathodal side (positions −60 to +60) during the first 10 minutes. FIG. 5F illustrates cross-correlation analyses comparing the averaged fluorescence and membrane speeds (FIGS. 5D-5E) at the anodal and cathodal sides of the cell. FIG. 5G illustrates a cross-correlation analysis was performed using the Galvanin-GFP fluorescence and membrane velocities averaged across all 46 individual cells.

FIGS. 6A-6D illustrate a cell separation device for genome-wide CRISPRi screen. FIG. 6A illustrates schematics of an assembled galvanotaxis screen device. FIG. 6B illustrates a rendered image of a cross-section of screen device. FIG. 6C illustrates finite element analysis using COMSOL. FIG. 6D illustrates a summary of genome-wide library screen targeting 18,901 genes for knockdown.

FIGS. 7A-7E illustrate an example cell separation device for secondary, focused CRISPRi screen. FIG. 7A illustrates schematics of an implementation of the galvanotaxis screen device. FIG. 7B illustrates a rendered image of a cross-section of the screen device. FIG. 7C illustrates an example migration module insert. FIG. 7D illustrates finite element analysis using COMSOL Multiphysics software used to determine the electrical environment at the surface of the track-etch membrane. FIG. 7E illustrates quantification of average speed along the electric field direction during migration in a collagen gel with exposure to an electric field (300 mV/mm).

FIGS. 8A-8F illustrate that the sensory mechanism of Galvanin depends on a highly charged ectodomain. FIG. 8A, in the left column, illustrates example micrographs of Galvanin-GFP in individual cells exposed to an electric field for 10 minutes. FIG. 8A, in the right column, illustrates semilogarithmic plots of corresponding fluorescence profiles for the examples shown. FIG. 8B illustrates a summary of vE/D values across the different electric field strengths. FIG. 8C illustrates estimated ectodomain charge based on the vE/D values of FIG. 8B. FIG. 8D, in the left column, illustrates a schematic illustrating the model of Galvanin electrophoresis due to the net negative charge. FIG. 8D, in the right column, illustrates the ectodomain was altered using engineered GFP proteins with either a strongly negative charge (−42e net ectodomain) or weak charge (+9e net ectodomain charge). FIG. 8E illustrates representative fluorescence micrographs of cells expressing the engineered Galvanin constructs exposed to a 300 mV/mm electric field. FIG. 8F illustrates compass autocorrelation of cells migrating in a collagen gel.

FIG. 9A-9E illustrates that galvanin exhibits rapid membrane diffusion consistent with a single-pass transmembrane protein. FIG. 9A illustrates an example fluorescence micrograph of a cell exposed to an electric field (300 mV/mm) for 5 minutes. FIG. 9B illustrates plots that show the decay in fluorescence intensity at the anodal side of the cell once the electric field is turned off, corresponding to a re-equilibration of Galvanin throughout the plasma membrane. FIG. 9C illustrates the squared width S2, which was used as a measure of the increase in the mean-squared displacement of the fluorescence signal over time. FIG. 9D illustrates an estimation of diffusion coefficient. FIG. 9E illustrate histograms summarizing average speed along the electric field direction in the Galvanin knockout cell line, expressing Galvanin-GFP, −42GFP-Galvanin, or +9GFP-Galvanin.

FIGS. 10A-10C illustrate CRISPR interference screen identifies Galvanin as a putative electric field sensor for directed cell migration. FIG. 10A, in the left column, illustrates a schematic of galvanotaxis screen. FIG. 10A, in the right column, illustrates a fraction of cells collected in the bottom reservoir after two hours. FIG. 10B illustrates a summary of focused library screen targeting 1,070 genes for knockdown. FIG. 10C illustrates fluorescence micrographs show rapid localization of Galvanin (TMEM154)-GFP toward the anodal pole when differentiated HL-60 neutrophils are exposed to an electric field (300 mV/mm).

FIGS. 11A-11E illustrates that galvanin is critical for persistent cathodal migration in HL-60 neutrophils. FIG. 11A illustrates that nuclear tracking was performed during migration of HL-60 neutrophils in a collagen gel exposed to an electric field (300 mV/mm). FIG. 11B illustrates average speed calculated based on nuclear tracking with 3 minute time intervals. FIG. 11C illustrates a schematic showing calculation of directed speed along the electric field vector. FIG. 11D illustrates histograms summarize average speed along the electric field direction. FIG. 11E illustrates that longer-term directed movement was determined by defining a compass autocorrelation.

FIGS. 12A-12C illustrate an overview of an example device designed to assay chemotaxis and chemokinesis. FIG. 12A illustrates a schematic of design to assay chemotaxis and chemokinesis. FIG. 12B illustrates a schematic of the insert that fits into the device in FIG. 12A. This contains a cell-porous membrane (e.g., 20 μm thick track-etch membrane with 3 um diameter pores that the cells will migrate through) that cells are placed on top of and migrate through. FIG. 12C includes an illustration showing the ability to place multiple devices from FIG. 12A in series.

FIGS. 13A, 13B illustrate a schematic of methodology to assay 3D migration in extracellular matrix. FIG. 13A illustrates a schematic of the multilayer extracellular matrix generated to assay 3D amoeboid migration. FIG. 13B includes an illustration of the 3D amoeboid migration assay.

FIGS. 14A, 14B illustrate an overview of device designed to assay galvanotaxis. FIG. 14A illustrates a schematic of optimized design to assay galvanotaxis. FIG. 14B illustrates a schematic of the insert that fits into the device in FIG. 14A. This contains a cell-porous membrane (e.g., 20 μm thick track-etch membrane with 3 μm diameter pores that the cells will migrate through) that cells are placed on top of and migrate through.

### DETAILED DESCRIPTION

Various implementations described herein relate to methods and devices that enable massively parallel (and scalable) separation of cells based on their migratory capabilities. They allow assessment of different ‘modes’ of cell migration (chemotaxis, chemokinesis, 3D amoeboid migration, galvanotaxis), but can also be considered as tools for selective separation or herding of cells. Some implementations allow spatial separation of tens of millions of cells, which is particularly important for human genome-scale experimentation.

Various implementations of the present disclosure can be utilized to perform diagnostic screening. For example, certain cancer cells may exhibit different migratory capabilities, and may respond to different stimuli, than other types of cells. In some implementations described herein, cells in a tissue sample (e.g., tissue biopsy obtained from a patient) can be analyzed and classified. For example, if the tissue sample includes cells that exhibit a particular migratory behavior in response to a particular stimulus, it may indicate that the cells are of a particular cell type. In some examples, these techniques can be used by pathologists or other clinicians to classify cell types in patient samples.

The present disclosure includes an Experimental Example that describes the identification of Galvanin, a previously uncharacterized membrane protein, that acts as an electric field sensor and mediates cathode-directed cell migration for human neutrophils. While past work has demonstrated that many plasma membrane proteins undergo electrophoresis across cells exposed to an electric field (G. M. Allen, supra; A. Sarkar, et al., Journal of Theoretical Biology 478, 58-73 (2019); Y.-J. Huang, et al., Journal of Cell Science 130, 2459-2467 (2017); L. Huang, et al., Journal of Cellular Physiology 219, 162-172 (2009); M. Poo, Annu Rev Biophys Bioeng 10, 245-276 (1981); B. M. Kobylkevich, et al., Physical Biology 15, 036005 (2018)), it has been challenging to connect bulk protein electrophoresis to galvanotaxis or to directed migration at the whole-cell level (E. I. Finkelstein, et al., Cell Motility and the Cytoskeleton 64, 833-846 (2007)). The Experimental Example provides a demonstration that electrophoretic relocalization of a single protein based on the net charge of the ectodomain is necessary and sufficient to reorient directed cell migration represents the first description of an electric field sensor of this class. Galvanin's electrophoretic response is rapid, enabling migrating cells to quickly repolarize within minutes of exposure to an electric field. For the rapidly migrating neutrophil cell type considered here, this time scale is consistent with the rapid response at sites of tissue injury. In this context, the wound-induced electric field would provide an additional layer of directional information, alongside other well-characterized chemical guidance cues involved in wound repair (Zhao 2022, supra).

In neutrophils, Galvanin accumulates at the anodal side, which becomes the cell rear. The close coupling observed between Galvanin relocalization and changes in local cell protrusion and retraction behavior indicates that Galvanin may steer cell migration either by locally activating retraction (for example, by stimulating myosin II contractility) or by locally inhibiting protrusion (for example, by inhibiting activity of the Arp2/3 complex responsible for branched actin network growth). In canonical neutrophil chemotaxis, chemoattractant receptors (typically GPCRs) are uniformly distributed on the cell's plasma membrane, and ligand occupancy of the receptors is higher on the side of the cell closer to the source of the chemoattractant because of its concentration gradient (Petri and Sanz, Cell and Tissue Research 371, 425-436 (2018); K. F. Swaney, et al., Annual Review of Biophysics 39, 265-289 (2010)). Downstream signaling from the activated GPCRs leads both to stimulation of actin network assembly at the presumptive cell front and to stimulation of myosin II-based contractility at the cell rear (H. Meinhardt, Journal of Cell Science 112, 2867-2874 (1999); J. Xu, et al., Cell 114, 201-214 (2003)). Because of the strong positive feedback within the respective “frontness” and “backness” mechanochemical modules, and strong negative feeback between them (A. H. Chau, et al., Cell 151, 320-332 (2012); Y. Wang, et al., Cell Reports 3, 1607-1616 (2013); A. Hadjitheodorou, et al., Nat Commun 12, 6619 (2021)), as well as the mechanical influence of tension in the cell plasma membrane to enforce tight and near-immediate coupling of changes in protrusion at one end of the neutrophil to drive retraction on the opposite side and vice versa (A. R. Houk, et al., Cell 148, 175-188 (2012); T. Tsai, et al., Developmental Cell, 189-205 (2019)), local activation of retraction or local inhibition of protrusion by Galvanin could each be completely sufficient to reorient whole-cell polarity for directed migration of neutrophils.

Beyond the neutrophil-like cell type considered in the Experimental Example, transcriptional data of Galvanin indicates highest expression in immune cells and skin (GeneCards ID: GC04M152618), in line with the perceived importance of galvanotaxis during wound healing. As described in the Experimental Example, the ability to engineer Galvanin and alter its physical and electrical properties demonstrates a tunable biological system that can be used to provide control over directed cell movement.

Particular examples will now be described with reference to the accompanying figures. The scope of this disclosure includes individual examples described herein as well as any combination of the examples, unless otherwise specified.

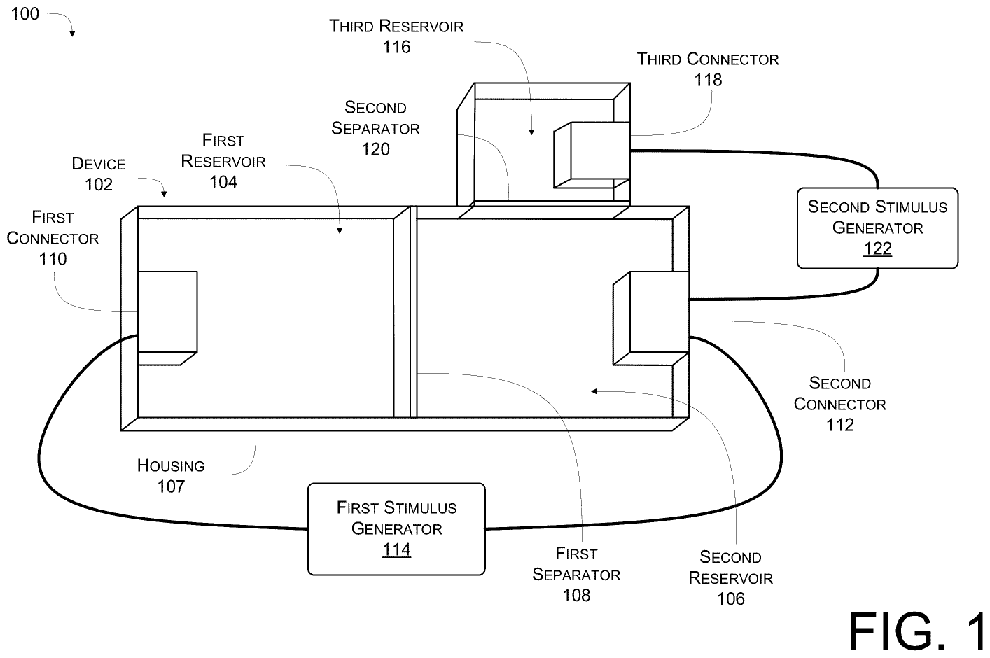

FIG. 1 illustrates an example environment 100 for causing and/or assessing cell migration. In various examples, the environment 100 includes a device 102 that includes a first reservoir 104 and a second reservoir 106. The first and second reservoirs 104 and 106 are, in various cases, configured to hold at least one solution containing cells. For instance, a housing 107 may at least partially enclose the first and second reservoirs 104 and 106. The housing 107, in some examples, represents boundaries (e.g., walls) of the first and second reservoirs 104 and 106. In various cases, the housing 107 may include a fluid-tight, biocompatible material that is compatible with cell culture methodologies. Examples of biocompatible materials include polymers (e.g., polylactic acid (PLA), polyethylene (PE), polypropylene (PP)), metals (e.g., stainless steel, titanium, etc.), glass, or the like.

In various implementations, a first separator 108 is disposed between the first reservoir 104 and the second reservoir 106. The first separator 108, in some examples, includes pores that are permeable to cells. The pores may have a width (e.g., a diameter) in a range of 1 micron (μm) to 15 μm. In various instances, the first separator 108 has a thickness in a range of 10 μm to 100 μm. The first separator 108 may also include a biocompatible material. In various examples, the first separator 108 is disposed in the housing 107.

In various implementations, it may be beneficial to separate cells based on their responses to a stimulus (e.g., galvanotaxis, chemotaxis, etc.). Various cells, including immune cells, endothelial cells, epithelial cells, fibroblasts, stem cells, and cancer cells, migrate in response to chemical and/or electric stimuli. For example, endothelial cells and epithelial cells may migrate in response to inflammatory signals associated with tissue repair and wound healing. Cancer cells, especially metastatic cells, may migrate in response to electric fields. Understanding stimulus-induced cellular migration can facilitate cell separation and can further enable the identification of new treatments for a variety of pathological conditions. For instance, identifying molecular markers associated with metastatic activity of cancer cells could provide new targets for cancer therapies.

Previously, chemotaxis was assessed using imaging. For instance, cells may be placed in a plate (e.g., an agar assay plate, a culture plate, or the like), and a chemical signal may be applied to one side of the plate. Cellular migration may be assessed, for instance, by imaging the plate using a microscope over a time period. While these methods facilitate small-scale measurement of cellular migration, they cannot accommodate studies of large cell populations, and they do not enable the identification of molecular markers associated with cellular migration. For instance, to determine the effect of a gene on cellular migration using current techniques, the gene could be knocked down in a cell population before assessment of chemotaxis. This approach is costly and time-consuming to screen a large number of genes for a single chemical signal. Further, current techniques focus on assessment of chemotaxis, rather than galvanotaxis. Accordingly, the interaction between chemotaxis and galvanotaxis cannot be measured using previously available techniques.

These issues can be addressed, in various implementations, by using the device 102, which can accommodate application of chemical signals and electric fields to cells. For instance, the device 102 may include conductive elements that contact cell media within the first and second reservoirs 104 and 106 to generate an electric field across the device 102. The conductive elements may be connected to a stimulus generator (e.g., a first stimulus generator 114) configured to generate a voltage between the conductive elements. In various implementations, the device 102 accommodates the removal of cells from the device 102 for genetic sequencing and/or other forms of analysis. Genetic sequencing can facilitate efficient characterization of large numbers of cells. Further, genetic sequencing enables the identification of molecular markers associated with cellular migration that were previously unknown.

In various implementations, the device 102 includes a first connector 110 connected to the first reservoir 104 and a second connector 112 connected to the second reservoir 106. The first connector 110 and/or the second connector are, in some examples, configured to output a differential stimulus across the device 102. In particular examples, the first connector 110 and the second connector 112 include electrodes that are electrically connected to the first reservoir 104 and the second reservoir 106, respectively. In some examples, the first connector 110 and the second connector 112 include agarose. For instance, the first connector 110 may include an agarose gel disposed between a first electrode and the first reservoir 104, and the second connector 112 may include an agarose gel disposed between a second electrode and the second reservoir 106. In some cases, the first connector 110 and the second connector 112 include one or more salts, such as sodium chloride, potassium chloride, disodium hydrogen phosphate, sodium dihydrogen phosphate, or the like. For instance, the agarose gels may include salts to enable conduction of an electric field through the agarose gels. In various instances, the agarose gels may be configured to reduce or prevent the transfer of toxic chemicals into the first and second reservoirs 104 and 106 and/or unintended reactions in the first and second reservoirs 104 and 106. For instance, the first and second connector 110 and 112 may include electrodes, and the agarose gels may prevent a pH change of a solution in the first and second reservoirs 104 and 106 (e.g., due to electrolysis, ion exchange, or the like).

The first connector 110 and the second connector 112 are, in some examples, connected to a first stimulus generator 114. The first stimulus generator 114 is configured to generate a differential stimulus between the first reservoir 104 and the second reservoir 106. In some examples, the first stimulus generator 114, in various cases, includes a power source configured to generate a voltage across the first connector 110 and the second connector 112, such that an electrical potential of a solution in the first reservoir 104 is different than an electrical potential in the second reservoir 106. In some examples, the first stimulus generator 114 includes an electromagnet configured to induce at least one magnetic field in the device 102, such as a magnetic field in the first reservoir 104 and/or the second reservoir 106.

In various implementations, the first connector 110 and/or the second connector 112 are configured to control a concentration of a chemical in the first reservoir 104 and the second reservoir 106. According to some cases, the chemical induces a response in at least one type of cell. For instance, the first stimulus generator 114 may be configured to store the chemical. The first stimulus generator 114 may include a pump configured to provide the chemical, via a tube, to the first connector 110. The first connector 110 includes, in some examples, an outlet configured to output the chemical into the first reservoir 104. In some examples, the second connector 110 may include or be connected to a pump. For instance, the second connector 110 may be configured to maintain a lower concentration of the chemical in the second reservoir 106 with respect to the first reservoir 104. Examples of chemicals include cell nutrients (e.g., glucose, amino acids, etc.), inflammatory signals (e.g., cytokines, chemokines, prostaglandins, histamine, reactive oxygen species, lactate, etc.), infection signals (e.g., formyl peptides, bacterial lipopolysaccharides, complement proteins, etc.), growth factors (e.g., transforming growth factor-β (TGF-β), vascular endothelial growth factor (VEGF), etc.), extracellular matrix (ECM) components (e.g., laminin, fibronectin, etc.) or the like. In some cases, by outputting the chemical in the first reservoir 104 and/or the second reservoir 106, the first stimulus generator 114 is configured to chemically induce migration in at least one type of cell in the first reservoir 104 and/or the second reservoir 106. For instance, the first stimulus generator 114 may be configured to output the chemical in the first reservoir 104 such that a concentration of the chemical in the first reservoir 104 is greater with respect to the second reservoir 106.

In some examples, the device 102 includes a third reservoir 116. The third reservoir 116, in various instances, is connected to a third connector 118. A second separator 120 may be disposed between the third reservoir 116 and the second reservoir 106. The second separator 120 may be different than the first separator 108 (e.g., have different size pores, include different materials, etc.). In some examples, the first separator 108 and the second separator 120 are the same. A second stimulus generator 122 may be connected between the second connector 112 and the third connector 118. In various examples, the second stimulus generator 122 is configured to output a differential chemical stimulus, induce a voltage, or output a magnetic field between the third reservoir 116 and the second reservoir 106. In some examples, the first stimulus generator 114 and the second stimulus generator 122 are configured to output the same signal. In some examples, the first stimulus generator 114 and the second stimulus generator 122 are configured to output different signals.

While FIG. 1 illustrates the second separator 120 disposed between the third reservoir 116 and the second reservoir 106, implementations of the present disclosure are not so limited. The second separator 120 may be disposed between the third reservoir 116 and the first reservoir 104 and/or the second reservoir 106. Accordingly, the second stimulus generator 122 is connected to at least two of the first connector 110, the second connector 112, and the third connector 118.

In various implementations, solutions in the first reservoir 104, the second reservoir 106, and the third reservoir 116 can be removed from the device 102 (e.g., aspirated, drained, etc.). Accordingly, the solutions can be processed (e.g., for cellular lysis, nucleic acid extraction, etc.) and/or applied to a sequencer. In some cases, the device 102 includes at least one outlet in the first reservoir 104, the second reservoir 106, the third reservoir 116, or any combination thereof.

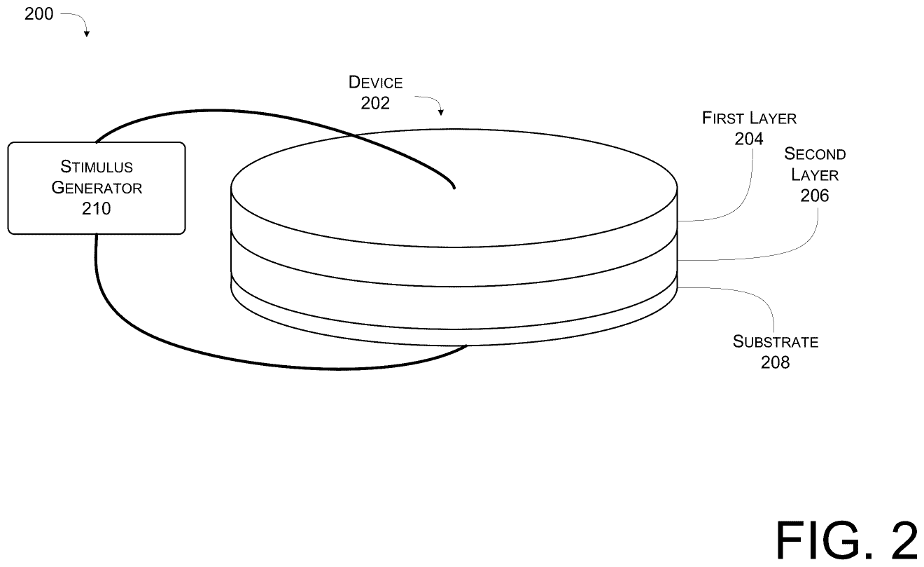

FIG. 2 illustrates an example environment 200 for causing and/or assessing cell migration. In various implementations, a device 202 includes a first layer 204 and a second layer 206. “Layer,” as used herein, may refer to a structure that retains its shape without an enclosure. A layer may be a gel (e.g., flexible) or a solid (e.g., inflexible). In some examples, the first layer 204 is disposed on the second layer 206. The first layer 204 and the second layer 206 include at least one of a protein, a tissue, an extracellular matrix (ECM) component (e.g., collagen, fibrin, elastic, laminin, proteoglycans, glycoproteins, etc.), or a porous membrane. For instance, the first layer 204 and the second layer 206 include at least one of collagen, fibrin, elastin, laminin, entactin, alginate, proteoglycans, glycoproteins, epithelial cells, endothelial cells, muscle cells, mucous cells, fibroblasts, adipocytes, another extracellular matrix component, or another cell type. In some examples, the first layer 204 is an ECM layer (e.g., including at least one ECM component), and the second layer 206 is a tissue layer (e.g., including cells). In some examples, the first layer 204 and the second layer 206 are ECM layers. In various implementations, proteins configured to facilitate cell adhesion to the layers may be disposed on the first layer 204 and/or the second layer 206. Examples of proteins configured to facilitate cell adhesion include arginine-glycine-aspartic acid (RGD) proteins, fibronectin, fibrinogen, vitronectin, collagen, or the like.

The second layer 206, in various examples, is disposed on a substrate 208. The substrate 208 is configured to hold cells. Accordingly, the substrate may be impermeable to cells. A stimulus generator 210 is, in some cases, connected between the first layer 204 and the substrate 208. As described above with reference to the first stimulus generator 114 in FIG. 1, the stimulus generator 210 may be configured to output a differential chemical signal between the first layer 204 and the substrate 208. In some cases, the differential chemical signal represents a difference in concentration of a chemical in the first layer 204 with respect to the substrate 208. The stimulus generator 210 may be electrically connected between the first layer 204 and the substrate 208 and configured to induce a voltage between the first layer 204 and the substrate 208. For instance, the stimulus generator 210 induces a difference in a relative electrical potential between the first layer 204 and the substrate 208.

In particular implementations, the first layer 204 or the second layer 206 is configured to be removed, dissolved, or dissociated to extract cells from the device 202. In various examples, the first layer 204 or the second layer 206 may be dissolved, for instance, using at least one of a chemical (e.g., a detergent, an enzyme, an acid, a base, or the like), heat, light, or physical methods (e.g., sonication, homogenization, freeze-thaw, or the like). In some examples, the first layer 204 can be removed from the device 202 (e.g., separated from the second layer 206). The first layer 204 may be removed and then dissolved in various cases.

For instance, a solution of cells may be applied to the substrate 208. In various cases, a portion of the cells may migrate from the substrate through the second layer 206 to the first layer 204 in response to the differential stimulus applied by the stimulus generator 210. The first layer 204 may include fibrin, and an enzyme (e.g., nattokinase) may be applied to the device 202 to dissolve the fibrin in the first layer 204. Accordingly, the portion of the cells may be removed from the device 202 and characterized (e.g., by genetic sequencing). In some examples, the second layer 206 may be removed from the device 202 and dissociated. The remaining cells in the second layer 206 may be characterized to, for instance, identify differences between the remaining cells in the second layer 206 and the portion of the cells in the first layer 204.

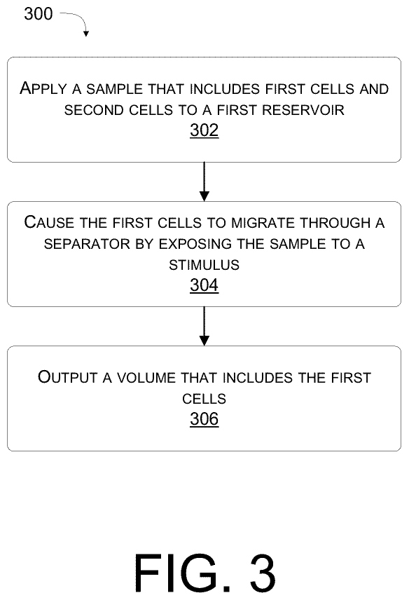

FIG. 3 illustrates an example process 300 for causing and/or assessing cell migration. In various implementations, the process 300 is performed by an entity, which may include one or more of a device (e.g., the device 102, the device 202), laboratory equipment (e.g., a polymerase chain reaction (PCR) machine, a thermocycler, a heating block, a microscope, etc.), a computing device, a user (e.g., a laboratory technician, care provider, or the like), or any combination thereof. According to some implementations, any of the steps of process 300 may be omitted.

At 302, the entity applies a sample that includes first cells and second cells to a first reservoir (e.g., the first reservoir 104, the substrate 208). In some examples, the first cells and the second cells have different responses to a stimulus (e.g., a chemical signal, an electric field, a magnetic field, etc.). The first cells and the second cells may be different cell types. In various cases, the first cells and/or the second cells are genetically modified. For instance, at least one gene may be inserted, modified, or knocked down in the first cells to generate the second cells. In some examples, the first cells and/or the second cells may be genetically modified immune cells, such as chimeric antigen receptor (CAR) T cells. In various cases, the first cells and/or the second cells may be genetically modified to treat a pathological condition. The first and second cells may be derived from a living organism (e.g., a mouse, a rat, a mammal, a non-human primate, a human, or another living subject) or from cultured cells.

At 304, the entity causes the first cells to migrate through a separator (e.g., the first separator 108, the second layer 206) by exposing the sample to the stimulus. The separator, in various cases, is configured to facilitate cellular migration from the first reservoir to a second reservoir (e.g., the second reservoir 106, the first layer 204). For instance, the separator may include pores that cells can migrate through. The first reservoir and/or the second reservoir, in various cases, are connected to a stimulus generator (e.g., the first stimulus generator 114, the stimulus generator 210). The stimulus generator is configured to apply a differential stimulus between the first reservoir and the second reservoir. For instance, the stimulus generator may cause a chemical to be pumped into the second reservoir such that a concentration of the chemical in the second reservoir is greater than a concentration of the chemical in the first reservoir. In some examples, the stimulus generator may be electrically connected to the first reservoir and the second reservoir and may induce a voltage between the first reservoir and the second reservoir.

At 306, the entity outputs a volume that includes the first cells. In some examples, the first reservoir and the second reservoir include cell media or another solution. The cell media can be extracted (e.g., aspirated, drained, etc.) from the second reservoir to isolate the first cells that have migrated from the first reservoir to the second reservoir. In some examples, the second reservoir includes a layer (e.g., a protein layer, a tissue layer, etc.) that can be removed, dissolved, or dissociated to isolate the first cells.

In various implementations, the entity may identify genetic characteristics of the first cells. For example, the first cells may undergo amplification (e.g., PCR, isothermal amplification, etc.) and a sequencing technique (e.g., Sanger sequencing, next-generation sequencing, third generation sequencing, RNA sequencing, exome sequencing, etc.) In some examples, the entity may output a second volume that includes the second cells. The genetic characteristics of the second cells may be determined and compared to the genetic characteristics of the first cells. For instance, based on sequencing, the entity may identify a first version of a gene (e.g., a wild-type gene) that is expressed by the first cells and a second version of the gene (e.g., a mutated gene) that is expressed by the second cells. In some examples, the gene may be associated with a particular pathological condition (e.g., a cancer type, a cancer subtype, an autoimmune disease, an autoimmune disease subtype, an inflammatory disease, an inflammatory disease subtype, a fibrotic disease, or a fibrotic disease subtype, etc.).

In some examples, the entity may identify a gene to induce or reduce cell migration of particular cells. For instance, identifying a gene (e.g., specific to the first cells or the second cells) to induce cell migration could have applications in the treatment of pathological conditions (e.g., increased immune cell migration, wound healing, reduced cancer cell metastasis, etc.), cell characterization and separation (e.g., identifying and isolating different cell populations), or the like. The devices, systems, and methods described herein can accommodate characterization of large volumes of cells (e.g., thousands, millions, tens of millions, greater than tens of millions, etc.) due to, for instance, the scalability of the device design and materials described herein.

In particular examples, the stimulus may include at least a portion of a treatment for a pathological condition. For example, the stimulus may include a chemical undergoing clinical testing for treatment of the pathological condition. Based on the genetic characteristics of the first cells and the second cells, the entity may determine an efficacy of the chemical to treat the pathological condition. For instance, the first cells may be cancer cells or infected cells, and the entity may determine that the first cells migrate toward the chemical, which may facilitate successful treatment when the chemical is paired with a cytotoxic agent.

In some examples, the entity may compare the genetic characteristics of the first cells and the second cells to a sample collected from a subject. For instance, the entity may compare genetic characteristics associated with a tumor biopsy collected from the subject to the genetic characteristics of the first cells and the second cells. Based on identifying similarities between the tumor cells of the subject and the first cells, the entity may identify a treatment predicted to successfully treat the cancer of the subject. In some cases, the subject may not be diagnosed with a particular pathological condition. Based on comparing the genetic characteristics associated with the sample collected from the subject to the genetic characteristics of the first cells and the second cells, the entity may determine that the subject has the particular pathological condition. Accordingly, the entity may provide an indication of follow-up tests to confirm the presence of the pathological condition.

In particular examples, the cells applied to the device may include cells collected from a subject. Based on determining the genetic characteristics of the first cells and the second cells of the subject, the entity may determine that the subject has a particular pathological condition and, accordingly, may provide a recommendation of follow-up tests to confirm the presence of the pathological condition.

The devices, systems, and methods described herein can be used, in various implementations, to identify molecular markers associated with cell migration (e.g., to identify molecular markers to induce cell migration), to identify disease targets for development of new treatments, to assess treatment efficacy, and to identify pathological conditions in subjects. The devices, systems, and methods described herein can be used for research, diagnostic, and therapeutic purposes.

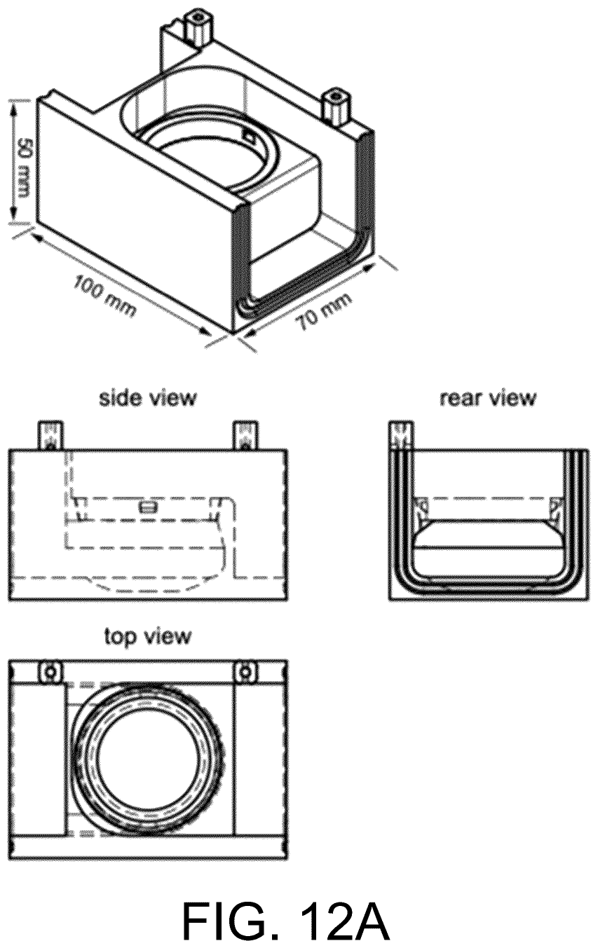

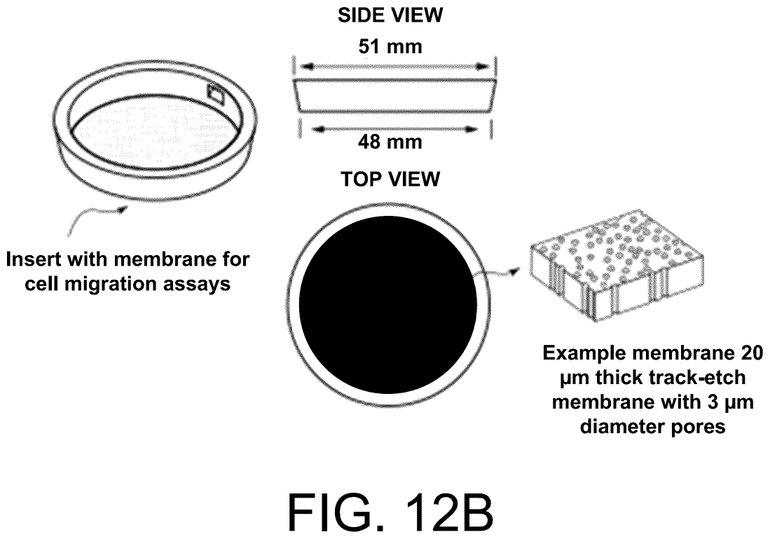

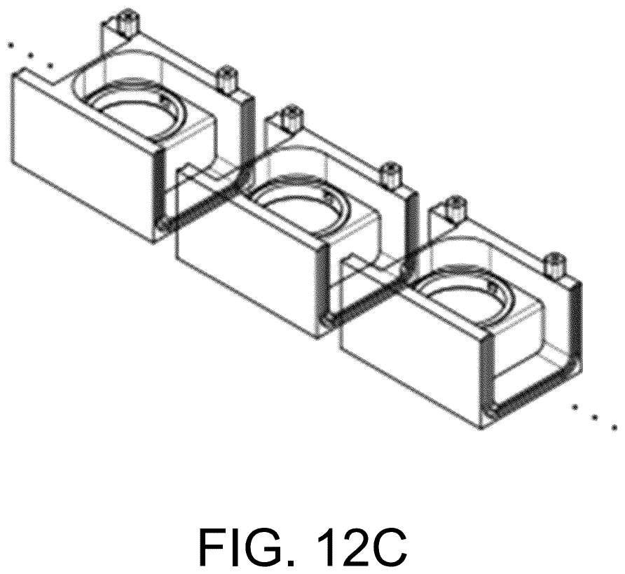

FIGS. 12A-12C illustrate an overview of an example device designed to assay chemotaxis and chemokinesis. FIG. 12A illustrates a schematic of design to assay chemotaxis and chemokinesis. Dashed lines indicate internal features not viewable in different perspectives. To make the device water-tight, the ends are clamped using flat plastic or metal end pieces and silicon gaskets. FIG. 12B illustrates a schematic of the insert that fits into the device in FIG. 12A. This contains a cell-porous membrane (e.g., 20 μm thick track-etch membrane with 3 μm diameter pores that the cells will migrate through) that cells are placed on top of and migrate through. In the case of chemotaxis, a chemoattractant is generally added to the reservoir below the cell-porous membrane. FIG. 12C includes an illustration showing the ability to place multiple devices from FIG. 12A in series.

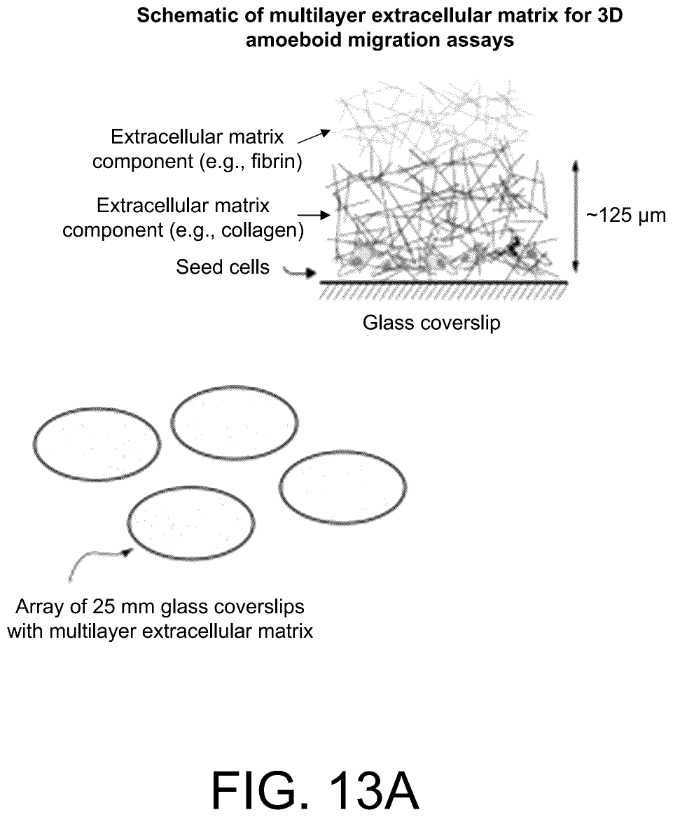

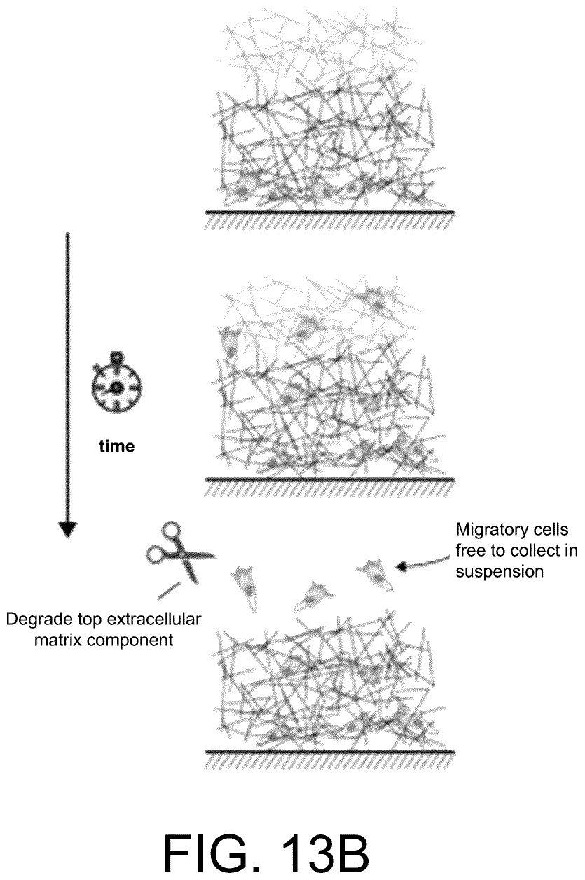

FIGS. 13A, 13B illustrate a schematic of methodology to assay 3D migration in extracellular matrix. FIG. 13A illustrates a schematic of the multilayer extracellular matrix generated to assay 3D amoeboid migration. Multiple extracellular matrix layers are sequentially generated on top of glass coverslips, with the cells added within the first later of extracellular matrix (e.g., composed of collagen). Two layers of extracellular matrix are shown (light and dark black lines). This assay is scaled up simply by preparing multiple coverslips in parallel. Individual coverslip preparations can be maintained together in larger tissue culture dishes during the migration assay. FIG. 13B includes an illustration of the 3D amoeboid migration assay. Cells are stimulated to migrate within the extracellular matrix over an appropriate amount of time. The most migratory cells will be more likely reach the second extracellular matrix layer (e.g., composed of fibrin). To recover this subpopulation of cells, the second extracellular matrix layer is selectively degraded, resulting in free-floating cells in suspension that can be collected for downstream analysis.

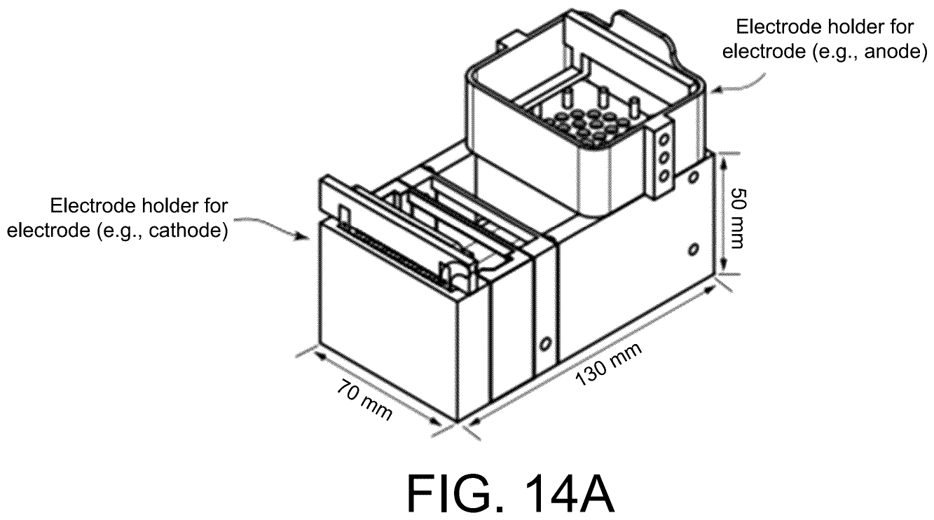

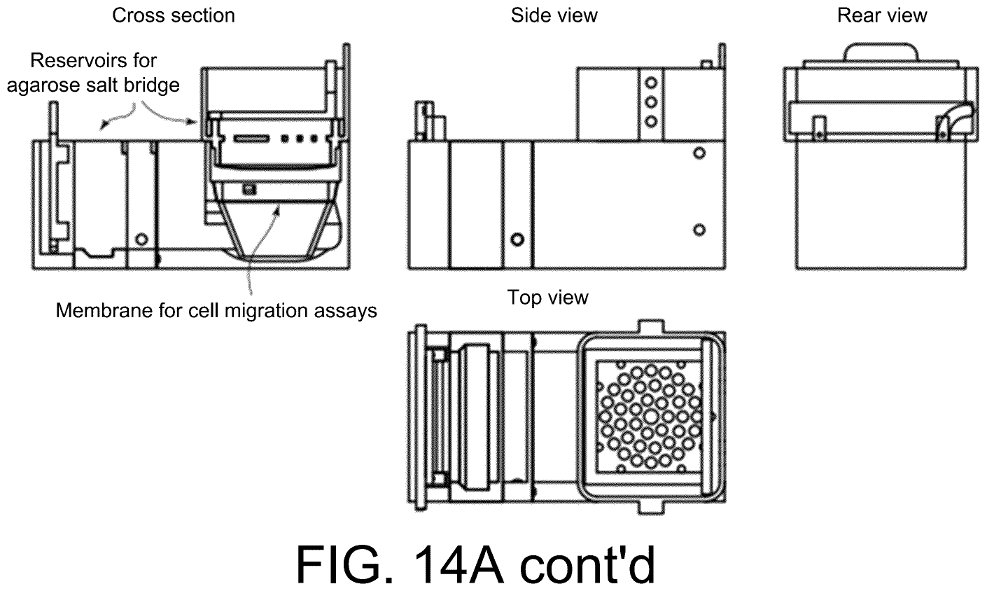

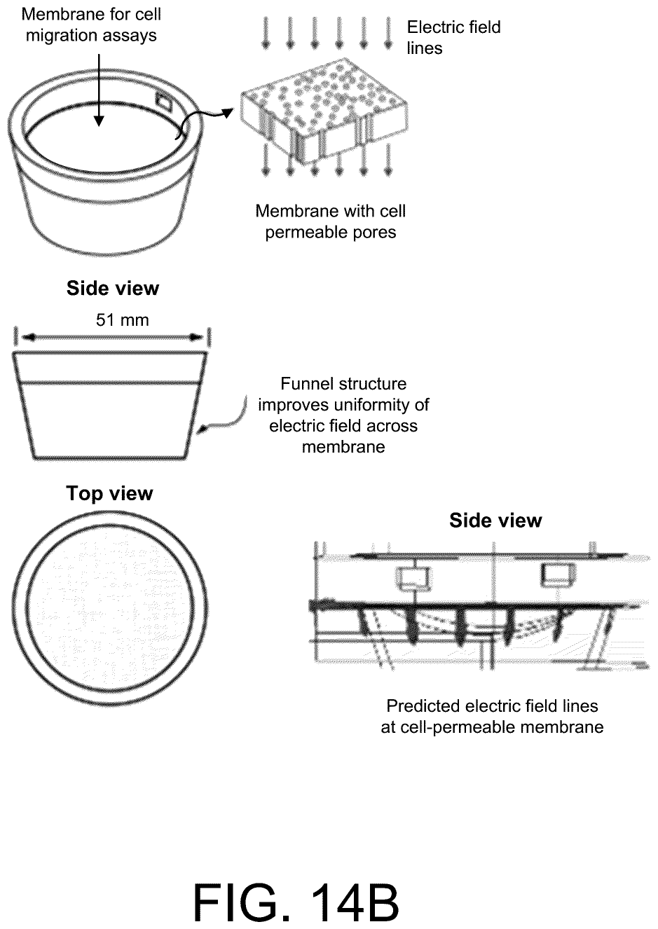

FIGS. 14A, 14B illustrate an overview of device designed to assay galvanotaxis. FIG. 14A illustrates a schematic of optimized design to assay galvanotaxis. Device was designed to provide a uniform electric field across the surface of a 47 mm diameter cell-permeable membrane. Components include two electrodes (e.g., silver chloride electrode), reservoirs to case agarose salt bridges that isolate electrodes from central region containing live cells. FIG. 14B illustrates a schematic of the insert that fits into the device in FIG. 14A. This contains a cell-porous membrane (e.g., 20 μm thick track-etch membrane with 3 μm diameter pores that the cells will migrate through) that cells are placed on top of and migrate through. An additional funnel structure improves the electrical properties, resulting in a uniform electric field across the surface of the membrane. This was demonstrated by modeling the device using finite element analysis. The bottom right image shows the results from this analysis, illustrating uniform electric field lines at the cell-permeable membrane where cells would be located.

Implementations of the present disclosure will now be described with reference to an Experimental Example.

### Experimental Example

### Materials and Methods

### Cell Culture and Neutrophil Differentiation.

Undifferentiated HL-60 cells were a gift from the lab of Dr. Orion Weiner (University of California San Francisco of San Francisco, CA). These cells were cultured and differentiated into HL-60 neutrophil-like cells as previously described (Millius and Weiner, Bacterial Transcriptional Control (2009), pp. 147-158; R. Garner, et al., Cytoskeleton. 77, 181-196 (2020)). Briefly, cells were cultured in RPMI 1640 medium containing L-glutamine and 25 mM HEPES 1640 (Gibco #22400089; Thermo Fisher Scientific of Waltham, MA) supplemented with 10% heat-inactivated fetal bovine serum (hiFBS, Gemini Bio Products #900-108; West Sacramento, CA), 100 U/mL penicillin, 10 μg/mL streptomycin, and 0.25 μg/mL Amphotericin B (Gibco #15240). Cells were maintained at 37° C. in 5% CO₂. Differentiated HL-60 cells were generated by incubating cells in media containing 1.57% dimethyl sulfoxide (DMSO, MilliporeSigma of Burlington, MA; #D2650;). Here, 1-1.5×10⁶ cells/mL were diluted by adding two volumes of additional media containing the DMSO. This culture media was replenished with fresh media, including DMSO, three days following the initiation of differentiation, and used in cell migration assays five days following the initiation of differentiation.

### Cloning and Cell Line Generation.

Pooled Plasmid Library Synthesis. All genomic integrations for the CRISPR interference (CRISPRi) work involved lentiviral transduction using undifferentiated HL-60 cells expressing dCas9-KRAB (pHR-UCOE-Ef1 a-dCas9-HA-2xNLS-XTEN80-KRAB-P2A-Bls) as previously described (Belliveau, supra). dCas9-KRAB is linked by a proteolysis-resistant 80 amino acid XTEN linker, driven by an EF1α promoter that was placed downstream of a minimal-ubiquitous chromatin opening (UCOE) element to prevent gene silencing and is based on a construct originally gifted by Dr. Marco Jost (Harvard Medical School of Boston, MA) and Dr. Jonathan Weissman (Massachusetts Institute of Technology of Cambridge, MA).

The genome-wide single-guide RNA (sgRNA) library was reported in Sanson et al. (K. R. Sanson, et al., Nature Communications 9, 1-15 (2018)) (Dolcetto CRISPRi library set A, Addgene #92385; Watertown, MA). This library contains 57,050 sgRNA, with 3 sgRNA per gene target and 500 non-targeting control sgRNA. For optimal library design, sgRNA were selected based on their position relative to annotated transcription start sites, expected on-target activity, and the presence of off-target matches.

The focused sgRNA library was constructed by taking the 1,070 most significant gene candidates identified in the initial screen. These were selected based on their adjusted p-value using the Benjamini-Hochberg procedure, with an arbitrary significant threshold set to allow selection of the desired number of genes. The sgRNA library was designed using a similar approach as the Dolcetto CRISPRi library, with 3 sgRNA per gene and then another 250 control sgRNA included from the original library. When selected individual sgRNA for gene targets included, those that appeared ineffective (e.g., individual log₂ fold-change values similar to the control sgRNA) were not included. In their place, an alternative sgRNA was selected from the Dolcetto CRISPRi library set B, which was also reported in Sanson et al. (supra). During filtering, genes specifically associated with transcription, translation and oxidative phosphorylation were also excluded. In addition, sgRNAs targeting the following genes were appended to the library: RICTOR, TRAF6, IFNAR2, PKN1, PRKCB, IL4R, HCAR2, B3GAT3, CHSY1, CHST9, CHST12.

The sgRNA library was synthesized as previously described (Sanson, supra). Briefly, oligonucleotides were synthesized to contain the sgRNA sequences, flanking BsmBI recognition sites, as well as primer sites for the initial amplification of the oligonucleotide library: 5′-[forward primer] CGTCTCACACCG (SEQ ID NO: 1) [sgRNA, 20 nt] GTTTCGAGACG (SEQ ID NO: 2) [reverse primer], where the forward primer and reverse primer sequences are AGGCACTTGCTCGTACGACG (5′-3′) (SEQ ID NO: 3) and ATGTGGGCCCGGCACCTTAA (5′-3′) (SEQ ID NO: 4), respectively (Twist Biosciences of South San Francisco, CA). The oligonucleotide library was resuspended in 10 mM Tris-CI, pH 8.5 to a concentration of 10 ng/μL. A 25 μL polymerase chain reaction (PCR) reaction was prepared, with 5 ng library DNA combined with 12.5 μL 2× NEBNext Ultra II Q5 Master Mix (#M0544S, New England Biolabs (NEB) of Ipswich, MA) and 0.5 μM of the forward and reverse primers noted above (Integrated DNA Technologies (IDT) of Coralville, IA). Library amplification was performed as follows: Step 1) Initial denaturation at 98° C. for 30 sec., Step 2) 10 cycles of denaturation at 98° C. for 10 sec.) and anneal/extension at 65° C. for 30 sec., and Step 3) a final extension at 65° C. for 5 min. Amplified DNA was purified using a QIAquick_PCR purification kit (Qiagen of Hilden, Germany; #28104) following protocol directions.

Next, the oligonucleotides were ligated into pXPR_050 (Addgene #96925) using a Golden Gate reaction (Engler and Marillonnet, DNA Cloning and Assembly Methods (Humana Press, 2014), pp. 119-131). The reaction was performed in 10 μL, containing 1 μL T7 10× StickTogether DNA Ligase Buffer, 0.75 μL BsmBI (10 U/μL, #FERER0451, Thermo Fisher Scientific), 0.5 μL T7 DNA ligase (#M0318S, NEB), 135 ng pXPR_050 DNA, 2.7 ng oligonucleotide insert DNA, and bovine serum albumin diluted to a final concentration of 100 μg/mL. This reaction was performed on a thermocycler: Step 1) 15 cycles alternating between 37° C. for 1.5 min. and then 25° C. for 3 min., Step 2) 50° C. for 5 min., and Step 3) 80° C. for 10 min. The DNA was purified using the QIAquick PCR purification kit and resuspended in 25 μL water. 5 μL of the ligation product was electroporated into 20 μL ElectroMAX Stbl4 electrocompetent cells (#11635018, Thermo Fisher Scientific). After a 90 minute outgrowth in SOC outgrowth media (#B9020S, NEB), cells were plated onto Bioassay LB agar plates (#12-565-224, Thermo Fisher Scientific) with 100 μg/mL carbenicillin. This was repeated six times total and colonies from the six plates were scraped to combine. Plasmid DNA was prepared using a plasmid maxiprep kit (performed by Genewiz from Azenta Life Sciences of South Plainsfield, NJ). Aliquots from each transformation were also used to estimate electroporation efficiency by plating out diluted aliquots onto 10 cm LB agar petri dishes containing 100 μg/mL carbenicillin. A total of 8.1 million unique transformants was estimated.

Cloning of individual sgRNA plasmids for CRISPRi. Individual sgRNA plasmids were constructed using sgRNA identified from the genome-wide CRISPRi screens as previously described (Belliveau, supra). Briefly, pXPR_050 was first linearized using the restriction enzyme BsmBI (New England Biolabs, #R0739S, which includes NEB buffer 3.1). Here, 20 μg of pXPR_050, 20 μL NEB buffer 3.1, and 10 μL BsmBI were combined for a 200 μL reaction and incubated for 5 hours at 55° C. The resulting linear pXPR_050 DNA was gel extracted using the QIAquick gel extraction kit (Qiagen, #28704) and resuspended in TE buffer (10 mM Tris-CI, pH 8.0; 1 mM EDTA) to a concentration of 10 ng/μL. The sgRNA inserts were generated by annealing complementary oligonucleotides with DNA overhangs compatible for ligation with the BsmBI-digested pXPR_050 DNA. The oligonucleotides were purchased as described below and annealed by combining 1.5 μL of each forward and reverse oligonucleotide (stock concentration of 50 μM in water), 5 μL NEB buffer 3.1, and 42 μL water. The mixture was first incubated for 5 minutes at 95° C. and then allowed to cool by lowering the temperature by 5° C. every 5 minutes until the sample was at room temperature. Finally to insert the sgRNA into the pXPR_050 vector, 1 μL of annealed sgRNA was combined with 20 ng of the BsmBI-digested pXPR_050 DNA and ligated using T4 ligase (NEB, #M0202S). The ligated DNA product was transformed into NEB Stable Competent E. coli (NEB, #C3040H) following manufacturer directions and successfully inserted sgRNA were confirmed by Sanger sequencing (performed by Genewiz from Azenta Life Sciences, NJ).

Forward oligonucleotides were 5′ CACCG(20 bp sgRNA target sequence)3′, while reverse complement oligonucleotides were 5′ AAAC(20 bp reverse complement sgRNA target sequence)3′ (IDT). The sgRNA target sequences used for individual CRISPRI knockdown cell lines are listed below.

Control sgRNA:
(SEQ ID NO: 5)
AGGGCACCCGGTTCATACGCNGG
TMEM154 (Galvanin) sgRNA:
(SEQ ID NO: 6)
GGGAACGAGCGCGATCACCA
UXS1 sgRNA:
(SEQ ID NO: 7)
GGTAGGGCCCTGGACCCGCG
GNPNAT1 sgRNA:
(SEQ ID NO: 8)
CGCGCCACAGTTGGGAACCG
VPS13B sgRNA:
(SEQ ID NO: 9)
CCAGGGACTTGGAGGTGGAG

Lentivirus Production for Stable Integration of sgRNA. For large scale lentivirus production (dCas9 construct or pooled sgRNA library), 15 μg transfer plasmid, 18.5 μg psPAX2 (Addgene #12260), and 1.85 μg pMD2.G (Addgene #12259) were diluted in 3.5 ml Opti-MEM I reduced-serum media (Gibco #31985070) and then combined with 109 μL TransIT-Lenti Transfection Reagent (Mirus Bio of Madison, WI, MIR6600). Following a 10-minute incubation, this mixture was added dropwise to confluent HEK-293T cells (American Type Culture Collection (ATCC) of Manassas, VA, CRL-3216) in a T175 flask containing 35 mL DMEM media (Gibco #11965-092) and supplemented with 1 mM sodium pyruvate (Gibco #11360-070). Lentivirus was recovered by collecting media 48 hours later, by centrifugation at 500 times gravity (g) for 10 minutes to remove any residual cells and debris. For the dCas9-KRAB construct, the lentivirus was additionally concentrated 60-fold using Lenti-X Concentrator (Takara Bio Inc. of Kusatsu, Japan, #631231).

For small scale lentivirus production (individual sgRNAs), lentivirus was prepared in 6-well tissue culture plates. Here 1 μg sgRNA transfer plasmid, 1 μg psPAX2, and 0.1 μg pMD2.G were diluted in 200 μL Opti-MEM I reduced-serum media and combined with 6 μL transIT. Following a 10-minute incubation, this mixture was added dropwise to confluent HEK-293T cells, with lentivirus collected as noted above.

Generation of CRISPRi Cell Lines. The dCa9-KRAB and sgRNAs constructs were integrated into undifferentiated HL-60 cells using a lentivirus spinoculation protocol. Briefly, lentivirus was added to 1 mL cells (1×10⁶ cells/mL) and polybrene reagent (final concentration of 1 μg/mL) in 24-well tissue culture plates. Cells were spun at 1,000 g for two hours at 33° C. The supernatant was removed and cells were placed in an incubator for two days prior to antibiotic selection for 6 days (dCas9-KRAB: blasticidin 10 μg/mL; sgRNA constructs: puromycin 1 μg/mL).

For CRISPRi sgRNA library preparations, lentiviral titers were estimated by titrating lentivirus over a range of volumes (0 μL, 75 μL, 150 μL, 300 μL, 500 μL, and 800 μL) with 1×10⁶ cells in a total of 1 mL per well of a 24-well tissue culture plate, using the spinoculation protocol noted above. Two days post-transduction, cells were split into two groups, with one placed under puromycin selection. After 5 days, cells were counted for viability. A viral dose that led to a 12.5% transduction efficiency was used for subsequent pooled library work. This low efficiency was targeted to ensure most cells only received one sgRNA integration.

Generation of Galvanin Knockout Cell Line via CRISPR-Cas9. Galvanin expression was disrupted by targeting exon 1 (containing the start codon and signal sequence) using CRISPR-Cas9, with clonal cell lines isolated to obtain cells with non-synonymous mutations. This was performed following a similar strategy to prior work in HL-60 cells (M. C. Gundry, et al., Cell Reports 17, 1453-1461 (2016)) and protocol recommendations using the Alt-R CRISPR-Cas9 system (IDT). Transfection was performed using the Neon Transfection System (Thermo Fisher, #MPK5000) using their 10 μL kit (Thermo Fisher, #MPK1096) as described below.

Briefly, the gene targeting crRNA (GGGAACGAGCGCGATCACCA (SEQ ID NO: 6)) was synthesized by IDT. The sgRNA was prepared by resuspending the crRNA and fluorescently tagged tracrRNA (Alt-R™ CRISPR-Cas9 tracrRNA, ATTO 488, 20 nmol; Ser. No. 10/007,810) in IDTE buffer (IDT, #11-04-02-01), each to a 200 μM concentration. These two RNA were annealed by diluting 2.2 μL of each crRNA and tracrRNA to a total volume of 10 μL in IDTE buffer and heating the sample to 98° C. for five minutes and allowing it to cool to room temperature on the bench top. The sgRNA-Cas9 ribonucleoprotein complex was then formed by first diluting 0.3 μL Alt-R Cas9 S.p. Cas9 Nuclease V3 (62 μM stock, IDT, #1081059) with 0.2 μL Resuspension Buffer R (from Neon 10 μL kit). This was combined with 0.5 μL of the annealed RNA sample and incubated at room temperature for 15 minutes. To prepare the final electroporation mixture, 1 μL of RNP complex was combined with 5×10⁵ wild-type HL-60 cells resuspended in 9 μL Buffer R and 2 μL of 10.8 uM Alt-R Cas9 Electroporation Enhancer (IDT, #1075916). Electroporation was performed with the Neon Transfection System according to manufacturer directions (electroporation settings: 1350 V, 35 ms pulse, 1 pulse). A non-sgRNA control was also included. Cells were then placed in 500 μL RPMI culture media containing 10% hiFBS for recovery. Transfection of the RNP was checked 48 hours later by flow cytometry and confirmed by an observed increase in fluorescence from the fluorescently tagged tracRNA.

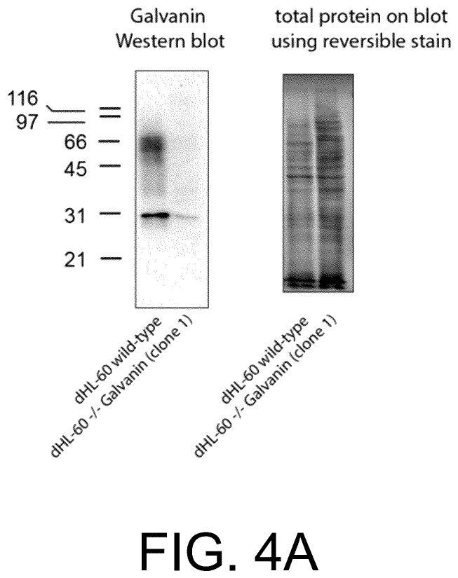

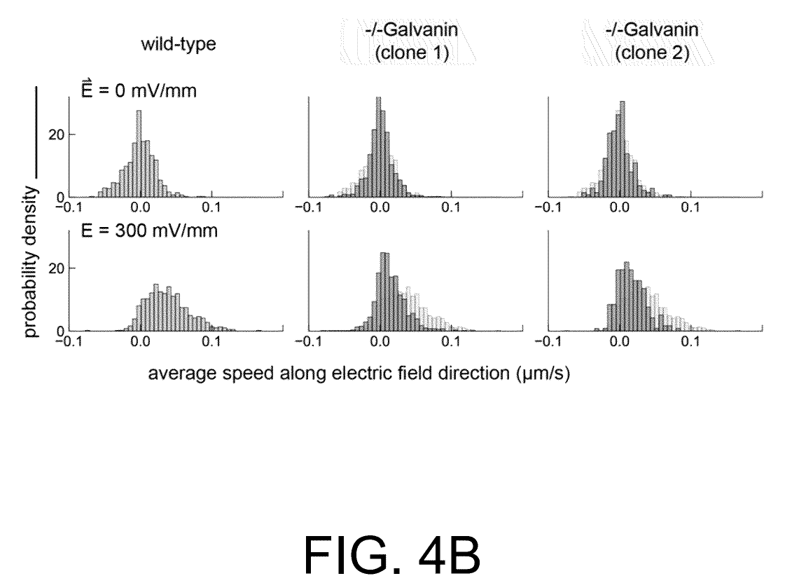

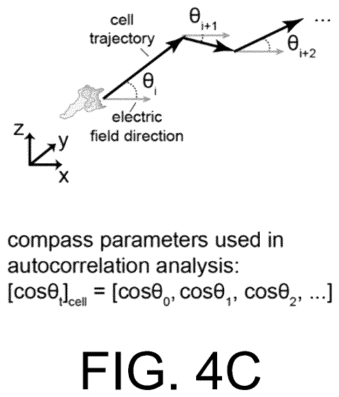

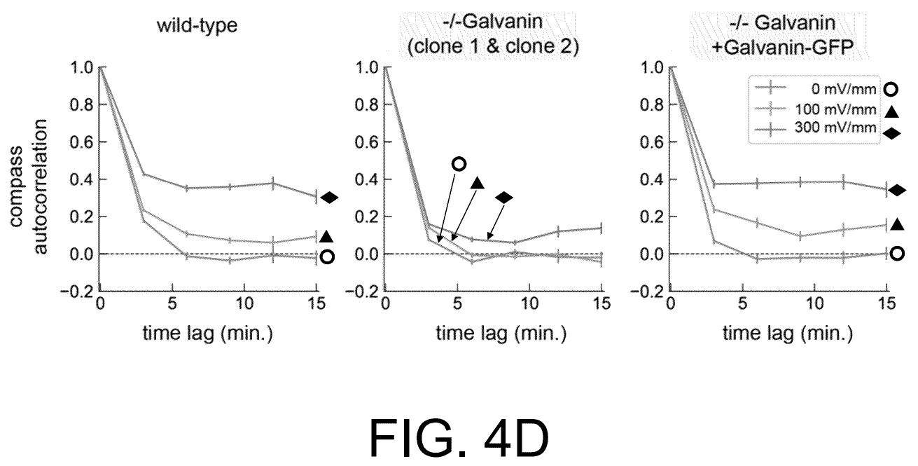

FIGS. 4A-4D illustrate validation of Galvanin knockout cell line and compass autocorrelation analysis. FIGS. 4A and 4B illustrate additional validation to confirm Galvanin gene disruption. FIG. 4A illustrates a western blot confirming loss of Galvanin protein expression. The left panel illustrates a blot using a polyclonal antibody against Galvanin. A near-complete loss of signal was found in the Galvanin knockout (clone 1) when compared to wild-type HL-60 neutrophils. The smear is expected from variable protein glycosylation. The right panel illustrates total protein stain on the blot using a reversible protein staining kit. Images are representative of Western blots performed in triplicate. FIG. 4B illustrates histograms summarize directed speed along the electric field vector during migration in a collagen gel. Comparisons are made between the HL-60 neutrophil wild-type cells (gray) and two clonal knockout lines. Clone 1 is used throughout the manuscript, while clone 2 was also genotyped to confirm gene disruption. Individual measurements represent the average speed along electric field direction, across 3 minute tracking intervals. Histograms present data from 200-900 cells and between 1600-7400 track intervals. FIG. 4C illustrates a schematic showing calculation of cosine θ, where θ is the angle between the cell trajectory (3 minute time interval) and the electric field vector. Autocorrelation is performed on the set of cosine θ values for individual cell tracks and then averaged across cells. FIG. 4D illustrates compass autocorrelation comparing the wild-type HL-60 neutrophils, −/− Galvanin knockout lines, and the genetic rescue (Galvanin-GFP expressed in −/− Galvanin knockout clone 1). For each cell line and electric field condition in FIGS. 4A and 4C, calculations were performed across 400-1000 cells, with approximately 30-150 cells per acquisition (wild-type: 4-6 acquisitions across different field strengths; Galvanin knockout: 7-8 acquisitions across different field strengths; Galvanin-GFP rescue: 6-9 acquisitions across different field strengths. However, for the −/− Galvanin knockout clone 2, data is from a single acquisition (0 mV/mm: 229 cells; 300 mV/mm: 197 cells). Error bars represent standard error of the mean in FIG. 4C.

Gene disruption was initially confirmed with the polyclonal cells through Sanger sequencing using the TIDE protocol (E. K. Brinkman, et al., Nucleic Acids Research 42, e168 (2014)), which estimates the distribution based on the read peak intensities, sgRNA target site, and known wild-type sequence. This indicates a high prevalence of single base-pair deletions (70%). Clonal isolates were then generated by limiting dilution into 96 well tissue culture plates and growth in RPMI media containing 50% hiFBS. Genotyping was determined by Sanger sequencing. The annotated exon 1 for TMEM154 (Ensembl of Hinxton, United Kingdom, ENST00000304385) is ACGTTTCAGAGAGGCTGCAGCCCGGCGCAGCATCCTGAGCGCGCCTCTGCCGAGGCGAG CGGACATGATGCAGGCTCCCCGCGCAGCCCTAGTCTTCGCCCTGGTGATCGCGCTCGTTC CCGTCGGCCGGG (SEQ ID NO: 10), where the italicized text indicates the coding sequence. Clone 1, used in the work of the main text, was found to have one allele with single base-pair deletion in the coding sequence ( . . . CCTGG-GATCGCGCTCGTTCCCGTCGGCCGGG (SEQ ID NO: 11)), where the dash indicates the deletion. The other allele contained two larger deletions, with the first spanning the starting codon (ACGTTTCAGAGAGGCTGCAGCCCGGCGCAGCATCCTGAGCGCGCC - - - CTAGTCTTCGCC - - - CTCGTTCCCGTCGGCCGGG (SEQ ID NO: 12)). Clone 2, a secondary cell line used to confirm loss of directionality in an electric field (FIGS. 4A, 4B) contained the same single base-pair deletion as clone 1, but at both alleles ( . . . CCTGG-GATCGCGCTCGTTCCCGTCGGCCGGG (SEQ ID NO: 11)).

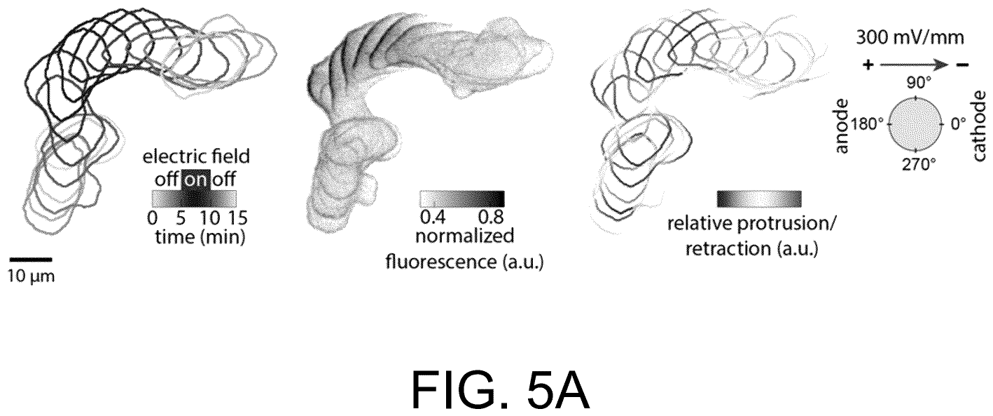

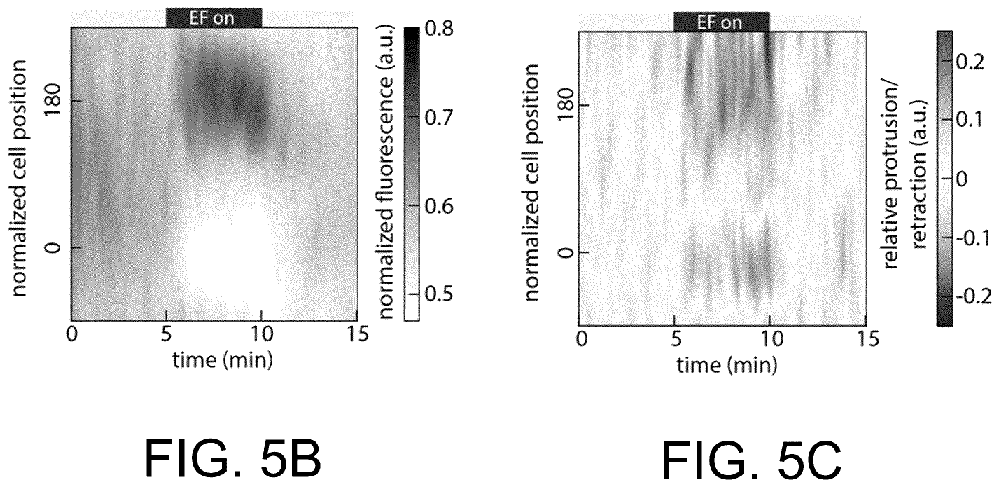

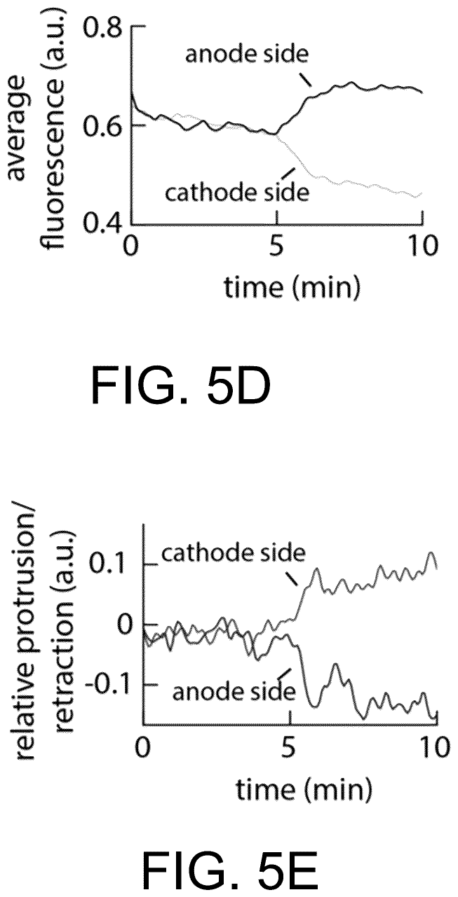

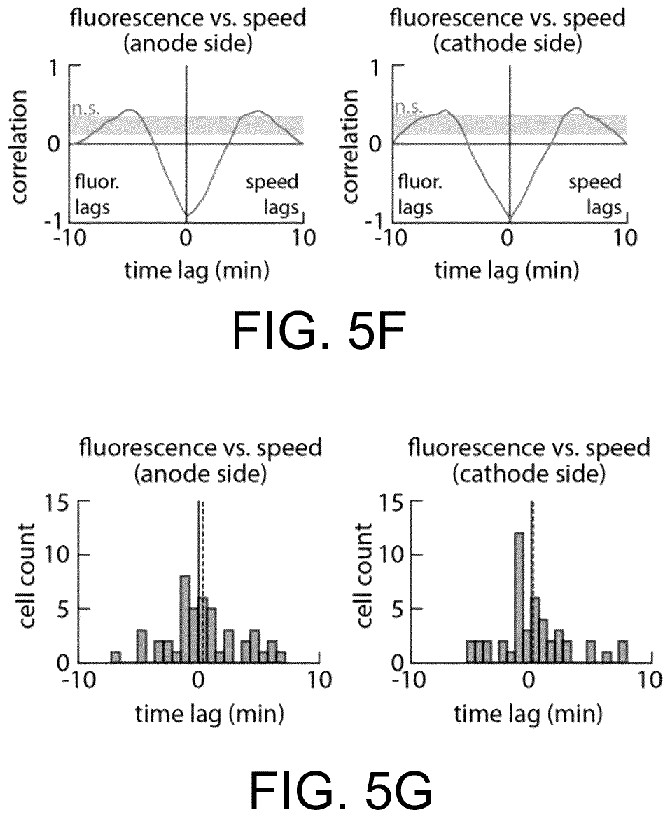

FIGS. 5A-5G illustrate that localization of Galvanin coincides with reduced local membrane speed and front-rear polarization. FIG. 5A illustrates an example analysis showing the temporal movement, Galvanin-GFP fluorescence intensity, and cellular protrusion/retraction activity of a single cell. Imaging was performed every 5 seconds over 15 minutes, with cells exposed to an electric field (300 mV/mm) between 5 minutes and 10 minutes. One-minute intervals are overlaid. FIG. 5B illustrates a kymograph showing the normalized Galvanin-GFP intensity, quantified at the cell periphery (in a band of 2 μm thickness) and averaged across 46 cells. Cell contours are defined with 360 positions, with position 0 corresponding to the right-most position relative to the cell centroid. FIG. 5C illustrates a kymograph quantifying membrane speed along the cell periphery, by comparing cell shape changes between 5 second intervals and correcting against bulk cell translocation. Values represent an average across the 46 cells considered in FIG. 5B. FIG. 5D illustrates averaged fluorescence at the anodal side (positions +120 to +240) and cathodal side (positions −60 to +60) during the first 10 minutes. FIG. 5E illustrates averaged membrane speed at the anodal side (positions +120 to +240) and cathodal side (positions −60 to +60) during the first 10 minutes. FIG. 5F illustrates cross-correlation analyses comparing the averaged fluorescence and membrane speeds (FIGS. 5D-5E) at the anodal and cathodal sides of the cell. Dash lines indicate the average minimum lag (0.1 minutes for the anode side and 0 minutes for the cathode side). The shaded region represents correlation values that are below a 99% confidence interval. FIG. 5G illustrates a cross-correlation analysis was performed using the Galvanin-GFP fluorescence and membrane velocities averaged across all 46 individual cells. Histograms show the lag corresponding to the minimum correlation value. Dashed lines near zero indicate the average lag value (anode side: 0.4 minutes; cathode side: 0.2 minutes).

Generation of Galvanin-eGFP Cell Lines. Constructs expressing Galvanin-eGFP and the charge-modified proteins were integrated by lentivirus follow the same approach as the CRISPRi work above, using vectors custom cloned by Epoch Life Sciences (Missouri City, Texas). The Galvanin (TMEM154) gene coding sequence (National Center for Biotechnology Information (NCBI) mRNA reference sequence NM_152680.3), with eGFP linked to the intracellular c-terminus using a GGGGSGGGGSGGGGSGS (SEQ ID NO: 13) amino acid linker sequence. This was driven from a spleen focus-forming virus (SFFV) promoter and included the native signal sequence. In the work looking at localization of Galvanin-eGFP and membrane protrusion/retraction activity (FIGS. 5A-5G), the construct was expressed in the CRISPRi cell line (only expressing dCas9-KRAB, without any sgRNA) with or without an additional myosin-mApple label (T. Y. C. Tsai, et al., Developmental Cell 49, 189-205.e6 (2019)). A second version of the Galvanin-eGFP construct with an HA tag at the c-terminus was also designed, with the sequence GGGGSGGGGSGYPYDVPDYA (SEQ ID NO: 14) appended after the eGFP sequence (bold indicates the HA tag). This was expressed in the Galvanin knockout cell line.

For the charge-engineered constructs, we removed the GGGGSGGGGSGGGGSGS (SEQ ID NO: 13)+eGFP sequence but kept the GGGGSGGGGSGYPYDVPDYA (SEQ ID NO: 14) HA tag sequence at the c-terminus. The entire extracellular domain between the signal sequence and transmembrane domain was replaced with the charged-GFP sequences (M. S. Lawrence, et al., J. Am. Chem. Soc. 129, 10110-10112 (2007)) and a linker sequence (-42e: XTEN linker, identical to that used in the dCas9 sequence; +9e: GGGGSGGGGSGGGG (SEQ ID NO: 15)) connecting to the native transmembrane sequence. The charged-GFP sequences were codon-optimized for expression in human cells.

Preparation of lentivirus and Integration into undifferentiated cells was performed identically to that described above for the CRISPRi cell lines using a spinoculation protocol. Enrichment of fluorescent-positive cells was achieved using fluorescence activated cell sorting (FACS, Sony Biotechnology Inc. of San Jose, CA, SH800).

### Genome-Wide and Secondary CRISPRi Assays.

Development of Device for Pooled Screens. The screen strategy involved exposing cells to an electric field, to drive their migration through the pores of a track-etch membrane. The aim was to isolate subpopulations where gene knockdown altered the ability of cells to sense the electric field and perform galvanotaxis. Enrichment or depletion of specific sgRNAs would be expected following enhancement or disruption of galvanotaxis, respectively. We have previously used track-etch membranes to perform screens of chemotaxis and undirected migration (e.g., chemokinesis) (Belliveau, supra). In order to scale up an in vitro galvanotaxis setup to allow exposure of millions of cells to an electric field, a device was designed for large scale galvanotaxis. Here, the initial focus was to ensure suitable exposure to an electric field, stable media conditions, and that the plastics and glue used in the device were non-toxic to cells. These aspects are described herein in the context of the devices used in the screen work. In general, conventions used in smaller scale galvanotaxis devices were followed, using a ‘salt bridge’ made of agarose to isolate the electrode chambers from the cell chamber. The device used in the secondary screen was modified following some computational analysis using the finite element analysis software COMSOL Multiphysics (v. 6.1, COMSOL Inc. of Stockholm, Sweden) to better understand the electrical properties.

The screen device was designed to be modular, enabling reuse of components such as the bulky center cell chamber, while making other components such as the track-etch membrane insert a one-time use item. Fusion 360 (Autodesk of San Francisco, CA) was used to design a device that could be printed using a 3D printer (UltiMaker S5 with Air Manager and Material Station; Ultimaker of Utrecht, Netherlands). All design files are available upon request. Transparent polylactic acid (PLA) filament (Ultimaker, #M-X6C-Y6J2) was used for each printed plastic component.

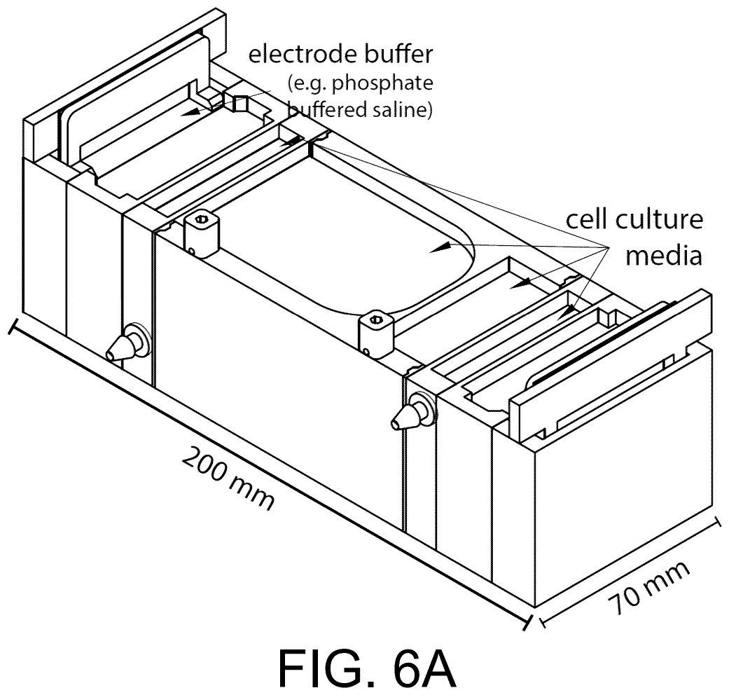

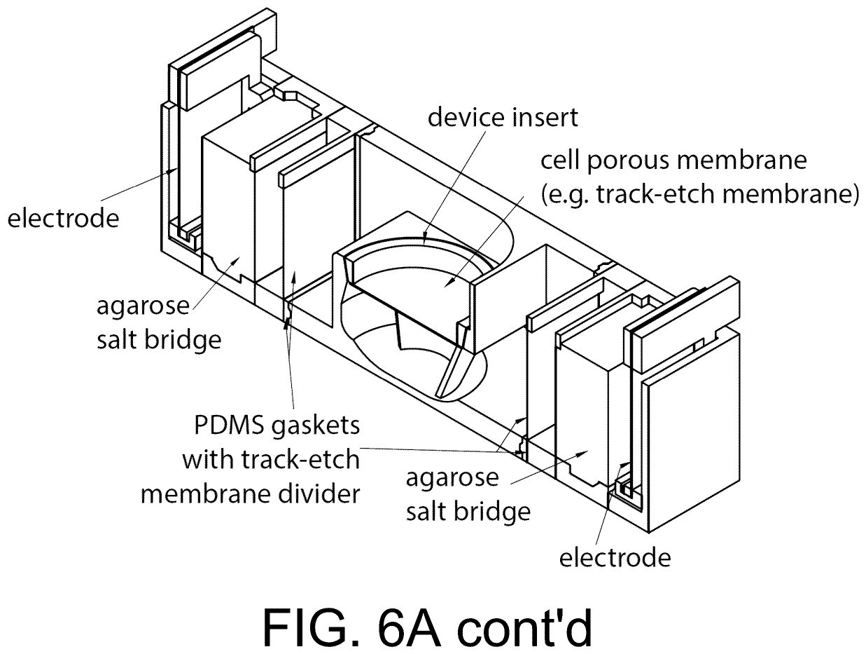

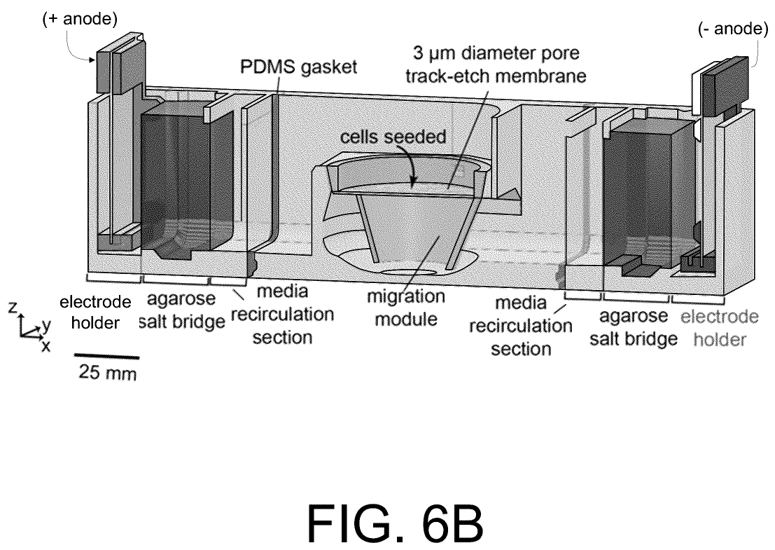

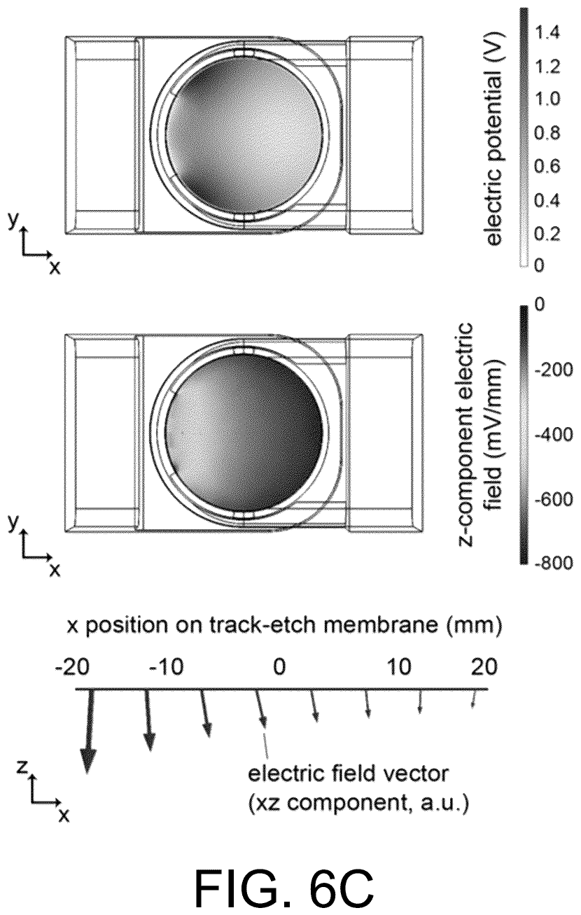

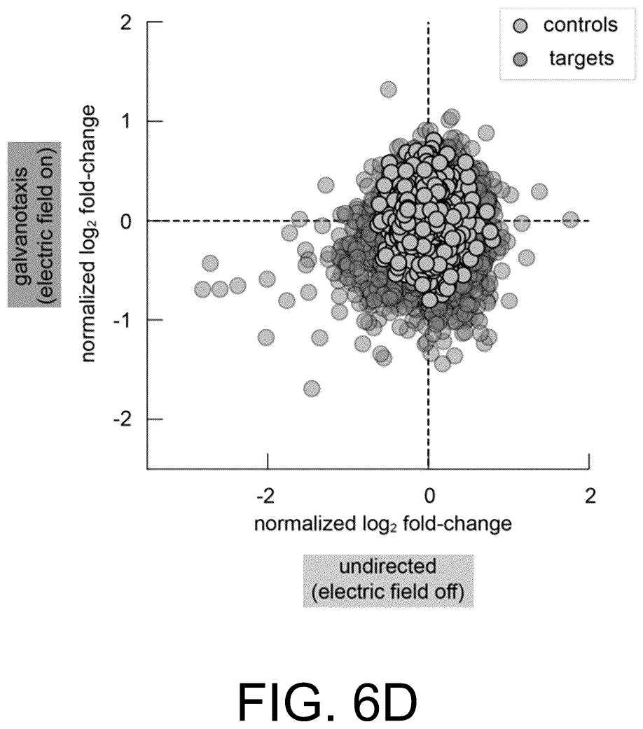

FIGS. 6A-6D illustrate a cell separation device for genome-wide CRISPRi screen. FIG. 6A illustrates schematics of an assembled galvanotaxis screen device. FIG. 6B illustrates a rendered image of a cross-section of the screen device. Components include Ag/AgCl electrodes and agarose salt bridges that isolate electrodes (in PBS) from region contain cells (in RPMI culture media with 5% hiFBS), and the center migration module containing a track-etch membrane (3 μm diameter pore size). Media was recirculated using the luer fittings connected to the ‘media recirculation section’, which recycled into a common beaker containing an additional liter of culture media. Colors are added to better distinguish different parts and features. FIG. 6C illustrates finite element analysis using COMSOL Multiphysics software was used to determine the electrical environment at the surface of the track-etch membrane. The top panel illustrates electric potential (voltage) across the surface of the membrane plane, taken relative to the smallest value calculated. The middle panel illustrates the Z-component of the electric field vector. The bottom panel illustrates a cross section at the plane of the track-etch membrane, showing the z-x component of the electric field vector (with the y-position centered on the membrane). FIG. 6D illustrates a summary of genome-wide library screen targeting 18,901 genes for knockdown. Data points show the normalized log 2 fold-change averaged across three sgRNAs per gene across independent experiments (5 screen replicates for galvanotaxis and 4 screen replicates for undirected migration). The undirected migration screen data comes from prior work (Belliveau, supra). Control values were generated by randomly selecting groups of three control sgRNAs.

FIG. 6A shows a rendering of the assembled device used in the genome-wide screen, while FIG. 6B shows a cross section. Each end contains a reservoir for Silver/silver chloride (Ag/AgCl) electrodes, immersed in a PBS solution. Ag/AgCl electrodes were generated by placing 20 gauge sheets of silver (Rio Grande of Albuquerque, NM, #101920) into a bleach solution for 30 minutes. During the screen, electrodes were connected to a direct current power supply (PowerPac 1000, Bio-Rad) via alligator clips, with a target total current of 400 mA. This resulted in joule heating of the cell culture media that was sufficient to heat it to 37° C., though we note that the total current was reduced during the experiment, if needed, to keep the temperature from going above 37° C.

Moving inward in the cross-section of FIG. 6B, the next segment is the agar salt bridge with 2% agarose in PBS. Between the salt bridges and central block is an auxiliary media reservoir that permits recirculation of culture media (with 5% hiFBS) utilizing a peristaltic pump. This reservoir is isolated from the central block with a track etch membrane, which aids in isolating the culture media in the central block from the agar bridges. Lastly, the central block where the cell migration experiments were performed contains the migration module that has the track-etch membrane. The track-etch membranes (3 μm diameter pores, shiny side oriented upward where cells are added; MilliporeSigma, #TSTPO4700) were adhered to printed plastic inserts with silicone adhesive sealant (LOCTITE SI 5011 CL non-corrosive RTV, Henkel of Düsseldorf, Germany #51387). During assembly, the mating surface of the migration module and its seat in the central block was coated with silicone high vacuum grease (DuPont; Fisher Scientific #146355D) to create a water-tight seal with the central block. The assembly was held together with a conventional bar clamp using two aluminum plates to distribute the clamping force. We used a custom molded PDMS gasket between the central block and the auxiliary media reservoirs.

The expected electric potential across the surface of the track-etch membrane and electric field strengths were calculated using the finite element analysis software COMSOL (FIG. 6C). A model of the fluid-filled region was generated using Autodesk Fusion 360 and imported into COMSOL. Analysis was performed using their electric currents module. Parameters included the temperature (293.15K) and media conductivity (1.27 S/m), which was experimentally measured for our cell culture media using a conductivity meter (Mettler SevenMulti, Mettler Toledo of Columbus, OH). In the analysis, the electric potentials and electric field strengths are based on a total current of 400 mA, taken to match the experimental conditions used.

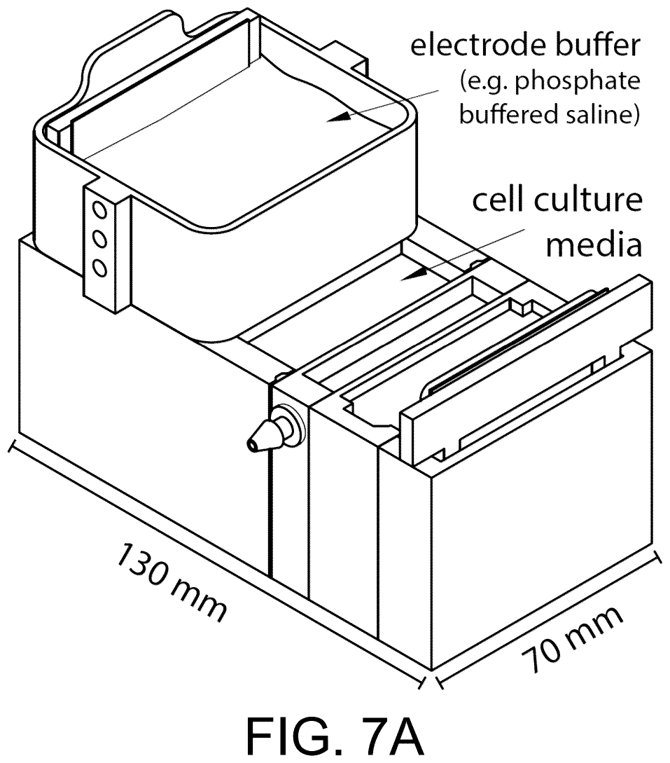

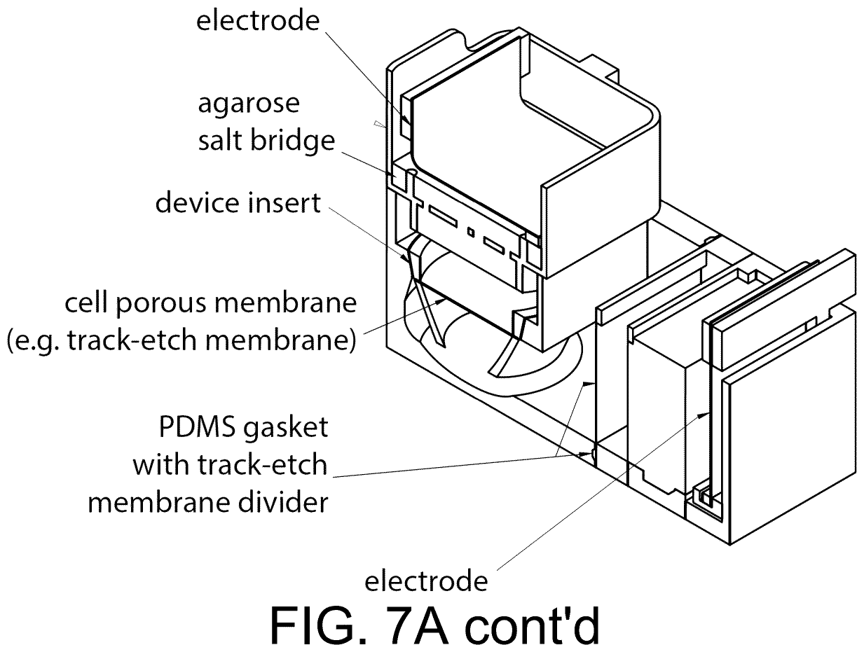

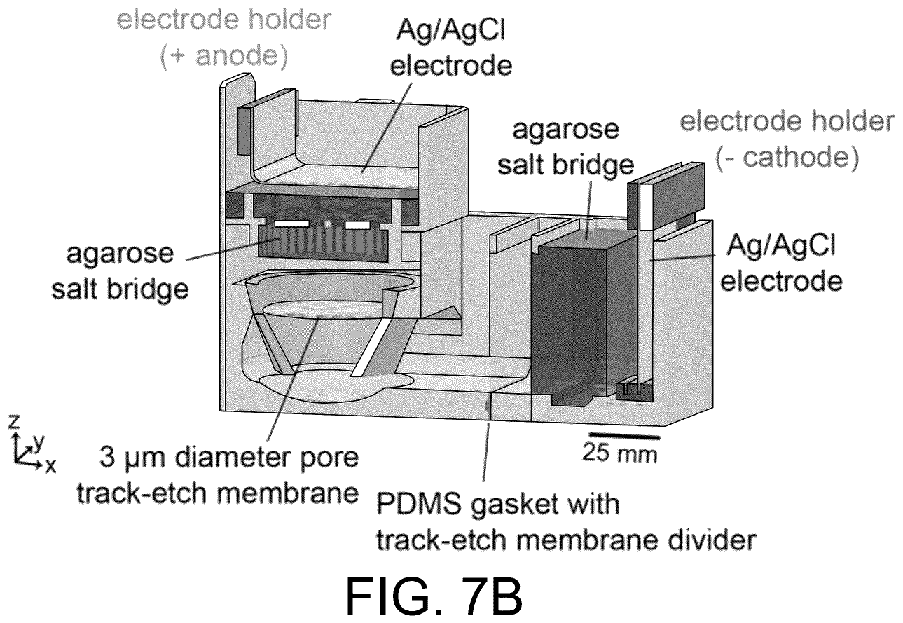

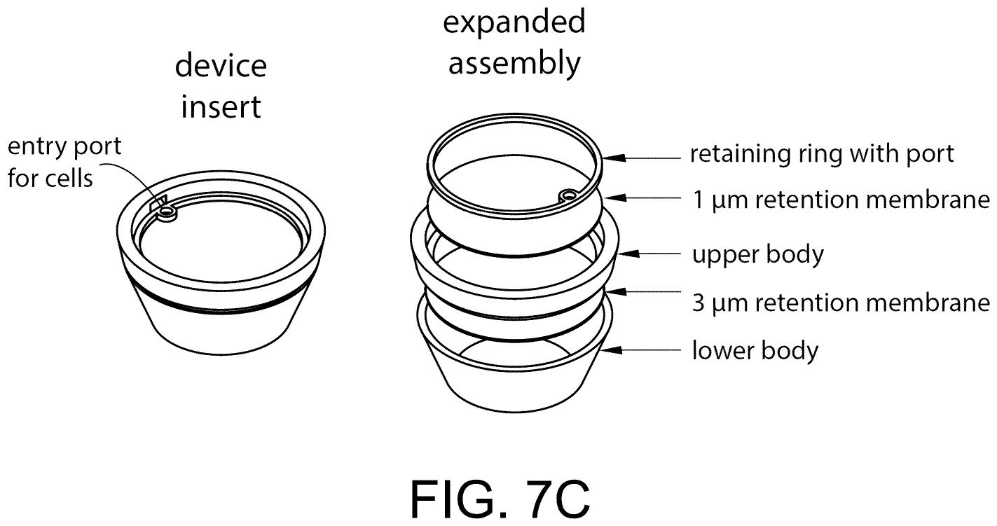

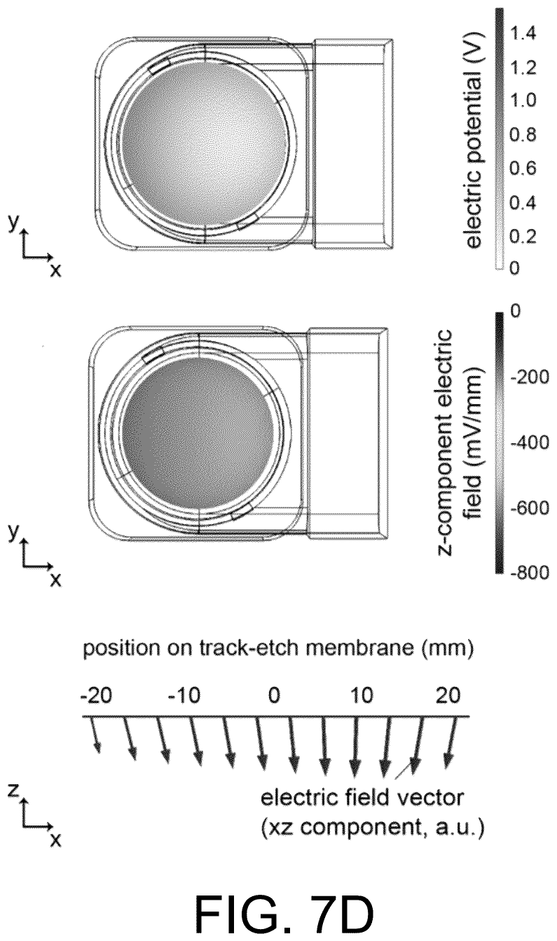

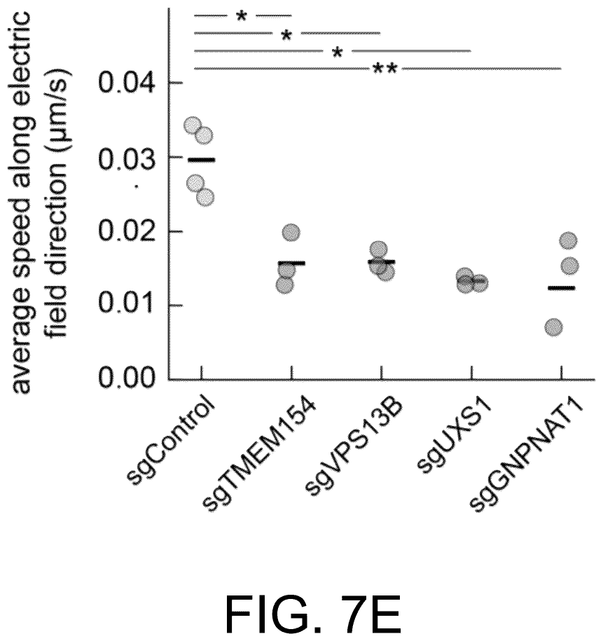

FIGS. 7A-7E illustrate an example cell separation device for secondary, focused CRISPRi screen. FIG. 7A illustrates schematics of an implementation of the galvanotaxis screen device. FIG. 7B illustrates a rendered image of a cross-section of the screen device. Components include Ag/AgCl electrodes and agarose salt bridges that isolate electrodes (in PBS) from region contain cells (in RPMI culture media with 5% hiFBS), and the migration module insert containing a track-etch membrane (3 μm diameter pore size). Peristaltic pumps were used to recirculate media into a beaker containing an additional liter of culture media, via the fittings shown, to help maintain uniform buffer conditions. Colors are added to better distinguish different parts and features. FIG. 7C illustrates an example migration module insert. The left panel illustrates a rendered drawing of assembled insert. The right panel illustrates that the different pieces shown in an expanded view. Each piece was printed separately and assembled with the track-etch membranes. The upper track-etch membrane with the retaining ring and cell seeding port was included to ensure the cells remained in place during handling of the device. FIG. 7D illustrates finite element analysis using COMSOL Multiphysics software used to determine the electrical environment at the surface of the track-etch membrane. This was done to improve the uniformity of the electrical environment relative to the device used in our initial screen. Plots show the (top) Electric potential (voltage) across the surface of the membrane plane, taken relative to the smallest value calculated, (middle) Z-component of the electric field vector, and (bottom) Cross section at the plane of the track-etch membrane, showing the z-x component of the electric field vector (with the y-position centered on the membrane). FIG. 7E illustrates quantification of average speed along the electric field direction during migration in a collagen gel with exposure to an electric field (300 mV/mm). Individual data points represent the average across cells from a single collagen preparation and the average is indicated by the horizontal line representing a total of 500-850 cells per cell line). A Tukey's range test was performed to make multiple comparisons across the cell lines, with only the sgRNA knockdown versus sgRNA controls found significant (**p-value<0.01).

The device used in the secondary screen is similarly rendered in FIG. 7A and a cross-section is shown in FIGS. 7A and 7B. While maintaining most of the features noted above, the orientation of the anode electrode holder was moved to be directly above the migration module. In this more recent implementation, we also modified our migration module, which is shown in FIG. 7C. Since the HL-60 neutrophils are poorly adherent, it was possible for cells to get swept away from the module. In this version, a second track-etch membrane (MilliporeSigma, 1.2 μm diameter, #RTTP14250) was included to ensure cells stayed within the migration module. Cells were loaded into the module via a small hole with a pipette which was then sealed with silicone vacuum grease. As shown in the COMSOL analysis (FIG. 7D), this new device geometry produced a more uniform electrical environment at the surface of the track-etch membrane.

Overview of Cell Collection and Experimental Replicates. For each CRISPRi cell migration experiment, cells were collected from three populations for gDNA extraction: On the day of each experiment, 5-day differentiated HL-60 neutrophil cells were collected and 3×10⁷ cells were set aside as a reference sample. The other two populations were the fraction of cells that migrated through the membrane and the fraction of cells that remained on top of the membrane.

Regarding experimental replicates, the genome-wide galvanotaxis experiment involved 5 experimental replicates. For the smaller scale CRISPRi screen, the galvanotaxis screen was performed 10 times, while the undirected migration screen was performed 8 times.

Cell migration screens identified gene candidates by comparing the number of cells that migrated through the track-etch membrane pores with respect to the reference sample, and those that did not with respect to the reference sample. This resulted in two separate measurements per migration experiment.

Removal of Cell Debris and Dead Cells Prior to Cell Migration Assays. Pooled CRISPRi libraries were differentiated in 15 cm dishes (55 ml cell culture per dish). Cellular debris and dead cells were removed from the differentiated HL-60 cell suspensions prior to use by density gradient centrifugation. Briefly, cells were first spun down (10 minutes at 300 g) and resuspended in 10 mL PolymorphPrep (Cosmo Bio USA of Carlsbad, CA; #AXS1114683), placed in the bottom of a 50 mL conical tube. Using a transfer pipette, 15 mL of 3:1 PolymorphPrep: RPMI media+10% hiFBS was gently layered on top by dispensing along the walls of the tube. This was followed by layering another 14 mL of RPMI media+10% hiFBS. Cells were spun at 700 g for 30 min with reduced acceleration and braking to reduce mixing. Live differentiated HL-60 cells were collected between the RPMI media and the 3:1 PolymorphPrep. RPMI media layers were diluted with one volume of RPMI media+10% hiFBS, and spun down once more for 10 minutes at 300 g. Finally, cells were resuspended in 10 ml RPMI media with 5% hiFBS and counted using a BD Accuri C6 flow cytometer (BD Biosciences of Franklin Lakes, NJ). Live cells were identified by their forward-scatter and side-scatter, which show a single population separate from dead cells or debris.

Cell Migration Screen Assay. Migration screens used track-etch membranes with 3 μm pore sizes as noted above in the device design. For each experiment, 20 million cells were added to the top of a track-etch membrane in the migration module. For undirected migration, devices were placed in a 37° C. incubator. For galvanotaxis screens, experiments were performed on the lab bench, with joule heating from electrical stimulation maintaining the media at 37° C. as noted above. Following incubation for the required time (8 hours for undirected migration and two hours for galvanotaxis experiments), the migration modules were removed from the central block. To ensure more complete recovery of the migratory cells, the bottom side of the track-etch membrane was gently scraped using a cell-scraper (Celltreat of Ayer, MA; #229310) to dislodge any cells remaining on the membrane surface. The migratory cells (bottom reservoir in central block) and retained cells (top of the migration module reservoir) were separately collected. Cells were spun down and washed with 1 mL PBS. Cells were spun down once more, with the PBS removed, and frozen at −80° C. for later genomic DNA extraction.

Quantification of sgRNA from CRISPRi libraries and Gene Identification. Genomic DNA (gDNA) was isolated using QIAamp DNA Blood Maxi (3×10⁷-1×10⁸ cells) or Midi (5×10⁶-3×10⁷ cells) kits following protocol directions (Qiagen, #51192 and #51183). gDNA precipitation was then used to concentrate the DNA. Briefly, salt concentration was adjusted to a 0.3 M concentration of ammonium acetate, pH 5.2 and 0.7 volumes of isopropanol were added. Samples were centrifuged for 15 minutes at 12,500 g, 4° C. Following a decant of the supernatant, the gDNA was washed with 10 mL 70% ethanol and spun at 12,500 g for 10 minutes, 4° C. The samples were washed in another 750 μL 70% ethanol, spun at 12,500 g for 10 minutes, 4° C., and decanted. The pellets were allowed to air-dry prior to resuspending them in water. The gDNA concentrations and purity were determined by UV spectroscopy.

The sgRNA sequences from each gDNA sample were PCR amplified for sequencing following protocols provided by the Broad Institute's Genetic Perturbation Platform (Broad Institute of Cambridge, MA). Briefly, gDNA samples were split across multiple PCR reactions, with 10 μg gDNA added per 100 μL reaction: 10 μL 10× Titanium Taq PCR buffer, 8 μL dNTP, 5 μL DMSO, 0.5 μL 100 μM P5 Illumina sequencing primer, 10 μL 5 μM P7 barcoded Illumina sequencing primer, and 1.5 μL Titanium Taq polymerase (Takara Bio, #639242). The following thermocycler conditions were used: 95° C. (5 minutes), 28 rounds of (95° C. (30 s)-53° C. (30 s)-72° C. (20 s)), and a final elongation at 72° C. for 10 minutes. PCR products (expected size of 360 bp) were gel extracted using the QIAquick gel extraction kit (Qiagen, #28704) following protocol directions. After elution, samples were further cleaned up using isopropanol precipitation. Here, 50 μL PCR DNA samples were combined with 4 μL 5M NaCl, 1 μL GlycoBlue coprecipitant (Thermo Fisher Scientific Technologies, #AM9515), and 55 μL isopropanol. Samples were incubated for 30 minutes and then centrifuged at 15,000 g for 30 minutes. The resulting pellet was washed twice with 70% ice-cold ethanol and resuspended in 25 μL of Tris-EDTA buffer. Illumina 150 bp paired-end sequencing was performed by Novogene Corp. (Sacramento, CA).

Sequence reads were quality filtered by removal of reads with poor sequencing quality and reads were associated back to their initial samples based on an 8 bp barcode sequence included in the P7 PCR primer. The 20 bp sgRNA sequences were identified and mapped to gene targets using a reference file for the genome-wide CRISPRi library (Sanson, supra). As noted in the overview section above, two log₂ fold-change values were calculated from the sequencing counts from each pooled screen experiment: each enriched sample (top or bottom reservoir collected cells) compared against the reference sample collected prior to the experiment. Since these two measurements are inversely correlated, the log₂ fold-change values from the less-migratory population collected in the top reservoir were multiplied by −1. This allowed the two sets of log₂ fold-change values to be compared directly, and all such measurements were averaged. Reported log₂ fold-changes represent averages across median-normalized replicate measurements from the multiple experiments performed. Here, a pseudocount of 32 was added to the sgRNA counts to minimize erroneously large fold-change values in cases of low library representation (F. Allen, et al., Genome Res. 29, 464-471 (2019)). Log₂ fold-changes were also scaled to have unit variance prior to averaging across individual experiments. P-values were determined by performing permutation tests (M. Ekvall, et al., Bioinformatics 36, 5392-5397 (2020)) between the calculated log₂ fold-change values for each gene target and our set of control sgRNA log₂ fold-change values. Adjusted p-values for multiple comparisons were determined using the Benjamini-Hochberg procedure (Benjamini and Hochberg, Journal of the Royal Statistical Society. 57, 289-300 (1995)).

### Image Acquisition.

All microscopy-based image acquisition was performed using microscope setups operated by MicroManager (v. 2.0) (A. D. Edelstein, et al., Journal of Biological Methods 1, (2014)). Details of the microscope configurations are provided below for each cell migration assay.

### Three-Dimensional Migration in Collagen Extracellular Matrices.

Experimental Setup. Cells were prepared for microscopy as previously described (Belliveau, supra; Garner, supra). Dead cells and debris were removed following the Polymorphprep protocol as described earlier. Briefly, following clean up, 3×10⁵ differentiated HL-60 cells were collected, resuspended in 1 mL L-15+10% hiFBS media containing 1 μg/mL DNA stain Hoechst 33324 and incubated at 37° C. for 15 min. During incubation with Hoechst stain, a 200 μL collagen aliquot was prepared: 6.5 μL 10× PBS, 12.5 μL 0.1 M NaOH, 111 μL L-15, and 20 μL hiFBS were combined with 50 μl 3 mg/mL collagen. The cell suspension was spun down and resuspended in the collagen mixture for a final concentration of 0.75 mg/mL collagen and then added to the channel of an Ibidi μ-Slide I (Ibidi of Gräfelfing, Germany, #80106). After 1 minute incubation at room temperature, the channel slide was inverted to help prevent cell sedimentation and incubated at 37° C. for gel formation. After 20 minutes, the collagen was set, and the channel slide was righted. The slide media reservoirs were each filled with 0.75 ml L-15 media containing 10% hiFBS. Imaging was performed within 30 minutes after the gel set.

Cells were exposed to an electric field using a custom device similar to that previously described (G. M. Allen, supra; B. Song, et al., Nat Protoc 2, 1479-1489 (2007)). A platform was designed that would hold the Ibidi sample slide and electrode reservoirs filled with a PBS solution and fit into a 96-well plate microscope stage insert. Salt bridges were 3D printed and filled with 2% agarose in PBS to connect the electrode reservoirs to the Ibidi media reservoirs. All device pieces were printed using transparent PLA filament (Ultimaker, #M-X6C-Y6J2), 3D files available upon request. Ag/AgCl electrodes were produced by immersing strips of silver foil (0.127 mm thick; Alfa Aesar of Haverhill, MA; Cat #11440-GW or 30-Ga 0.999 fine silver from Rio Grande, #101930) in bleach for 30 minutes and then rinsing several times with water and 70% ethanol. The electrodes were connected to a direct current power supply (Keithley Instruments of Cleveland, OH; #2200-72-1 or B&K Precision of Yorba Linda, CA; #BK9184B-ND) via alligator clips. The power supply was used in constant current mode to maintain a constant current density and voltage was measured using a multimeter by immersing platinum electrodes at each side of the Ibidi channel.

Cells were imaged at 37° C. on a Nikon Ti2 inverted microscope, equipped with a piezo-z stage (Applied Scientific Instruments of Eugene, OR; PZ-2300-XY-FT), a Yokogawa CSU-W1 spinning-disk confocal (Yokogawa of Tokyo, Japan), and iXon EMCCD camera (Andor of Belfast, United Kingdom). Imaging was performed with a 20× 0.95 NA water objective lens using sequential brightfield and epifluorescence illumination (405 nm laser; CSU-W1 Penta Dichroic; Emission: Chroma ET450/40m). For each sample, a 30 min time-lapse movie was acquired with 60 s intervals. A z-stack was acquired over 300 μm with acquisitions every 3 μm. In general experiments were performed over three different days using freshly prepared slides.

Cell Tracking and Quantification. Cell tracks were extracted from the DNA channel of time-lapse microscopy images using the TrackMate package (v. 7.12.2) (D. Ershov, et al., Nat Methods 19, 829-832 (2022)) in FIJI (v. 1.54) (J. Schindelin, et al., Nat Methods 9, 676-682 (2012)). Any stage drift was corrected using a small number of non-motile cells present in the collagen gel. These were taken as fiducial markers, with x, y, z drift correction performed to maintain their non-moving position. Cell track information, including position and time, was aggregated into a table using pandas Python package (v. 1.4.4) (W. McKinney, Proceedings of the 9th Python in Science Conference, 56-61 (2010)). For the Galvanin knockout expressing Galvanin-GFP (e.g., the genetic rescue line), cells were not sorted prior to these migration experiments, and there was a small cell subset (20%) that were not Galvanin-GFP positive. Prior to each acquisition, a single z-stack was taken of the GFP channel (488 nm), and this data was used to filter out non-fluorescent cells.

Cell migration speeds were calculated using 180 second intervals using the three-dimensional track vectors. Calculations were performed on an individual cell basis, with an average migration speed calculated across all time intervals in the 30 minute video. The average speed along the average speed along the electric field vector was similarly calculated, but in this case the x-component (i.e., electric field direction) of the track vectors was considered. Comparisons of the Galvanin knockout and Galvanin-GFP rescue with the wild-type cells were performed using the two-sided Mann-Whitney U nonparametric test scipy.stats.mannwhitneyuo in the SciPy Python package (v. 1.9.1) (SciPy 1.0: fundamental algorithms for scientific computing in Python | Nature Methods). Cell tracks longer than 16 minutes were included in this analysis.

The compass autocorrelation involved first calculating the cosine angle (cos θ) between the migration track vector and the electric field vector at each 180 second interval (see FIG. 4C for a schematic illustration). For each cell, this resulted in an array D(t) of cos θ values over the 30 minute video. A normalized non-overlapping autocorrelation was calculated by iterating over each possible time lag. Here the sum of products was computed between non-overlapping pairs of cos θ values,

$$S ( τ ) = {\sum }_{i = 0}^{N - τ - 1} D ( i \cdot ( τ + 1 ) ) \cdot D ( i \cdot ( τ + 1 ) + τ ) . \qquad ( 1 )$$

Normalization by the square root of the product of the sum of squares of the corresponding non-overlapping segments of the signal at the current and lagged positions was then performed,

$$Compass Autocorrelation ( τ ) = \frac{S ( τ )}{\sqrt{{\sum }_{i = 0}^{N - τ - 1} {D ( i \cdot ( τ + 1 ) )}^{2} \cdot {\sum }_{i = 0}^{N - τ - 1} {D ( i \cdot ( τ + 1 ) + τ )}^{2}}} . \qquad ( 2 )$$

Only long cell tracks (27 minutes or longer) were used in the autocorrelation analysis.

### Two-Dimensional Migration Under Agarose.

Experimental Setup. Cells were resuspended to a concentration of 2×10⁶ cells/ml in L-15 Medium with 10% hiFBS. With an ibiTreat μ-Slide-I (Ibidi, #80106) placed on a 37° C. heat block, 100 μL of cells were added to the channel and incubated for 5 minutes to allow them to sediment and loosely adhere to the bottom coverslip. Warm, liquid 2.5% UltraPure low melting point agarose (Invitrogen of Waltham, MA, #16520-050) in L-15 with 10% hiFBS was added to one channel reservoir and then centrifuged at 28-37° C. for 5 min at 700 g. This was performed in a custom 3D-printed swinging bucket rotor plate adaptor (design available upon request). Immediately after centrifugation, the slide was incubated on a 20° C. cooling plate (STIR-KOOL, Ladd Research of Essex Junction, VT; #SK-12D-AW) for 5 minutes to gel. Excess solidified agarose was observed in the media reservoirs of the Ibidi slide and removed with a pipet tip. L-15 media with 10% hiFBS was added to both reservoirs.

For under agarose experiments with Latrunculin A, cells were prepared as for other under agarose experiments, except prior to addition of cells to the μ-Slide I channel, 254 μm diameter 8 lb Maxima Ultragreen monofilament fishing line (West Marine of Seattle, WA) was threaded into the channel so that it stuck out on either end. After the agarose solidified, 25 μM Latrunculin A (Invitrogen, #L12370) in complete media was added to each reservoir. The monofilament line was removed, leaving a tunnel free of agarose the length of the Ibidi channel. Excess agarose was removed from the reservoirs. This thin tunnel, free of agarose, allowed flow of the media containing Latrunculin A along the channel length. This shortens the distance the drug must diffuse through the agarose.

Cells were imaged at 37° C. on an inverted microscope (Nikon Ti Eclipse; Nikon of Tokyo, Japan), with epifluorescence illumination using a Lambda 721 (721CUBE-480, Sutter Instruments of Novato, CA) and standard GFP filter cube (Chroma of Irvine, CA, ET-EGFP #49002; Ex: ET470/40×; Dichroic: T495LPXR; Em: ET525/50m). Imaging was performed with an oil 60× objective lens (Nikon 60× 1.4 NA plan apo phase contrast) using sequential brightfield and epifluorescence illumination and captured on an iXon EMCCD camera (Andor). For non-drug treated samples, imaging was performed at 5 second intervals, while for the Latrunculin A, imaging was performed at 10 second intervals. Exposure of cells to an electric field was performed as described in section ‘Three-Dimensional Migration in Collagen Extracellular Matrices,’ above. In this Example, a custom design was used where instead of the Ibidi slide, cells were added to a glass coverslip and overlaid with a 510 μm thick 1% agarose/L-15/10% hiFBS gel as previously described (R. Garner, et al., Cytoskeleton. 77, 181-196 (2020)). For this movie, imaging was performed using a 100× objective lens (Nikon 100× 1.45 NA plan apo phase contrast with 1.5× additional magnification applied).

### Quantification of Peripheral Galvanin-GFP Signal and Membrane Velocity.

Segmentation of cells was performed using the phase images. Here, an image that is just the agarose background was subtracted pixelwise from the phase image, then the contrast was adjusted by renormalization. A Sobel discrete differentiation operator was then applied to the image, allowing us to identify the cell edge using a threshold cutoff, which was used to generate a binary mask of each cell. The fluorescence images were flat field corrected. The resultant segmentation masks and fluorescence images were rotated 90 degrees to the left and input to the ADAPT Plugin in FIJI (v.1.111) (D. J. Barry, et al., Journal of Cell Biology 209, 163-180 (2015)), with smoothing sigma of 2, a 2 μm cortex, and with 8 erosions. The signal kymograph and the velocity kymograph were generated by averaging across kymographs from individual cells. Only cells that were in frame and had no collisions nor segmentation errors over the collection period were included in the analysis.

The cross-correlation was quantified using:

$$C ( τ ) = \frac{1}{m} {\sum }_{t = 0}^{m - 1} \frac{( GFP ( t ) - \overline{GFP} ) \cdot ( vel ( t + τ ) - \overline{vel} )}{{σ}_{GFP} \cdot {σ}_{vel}} , \qquad ( 3 )$$

which computes the correlation between the average Galvanin-GFP signal (GFP(t)) and membrane velocity (vel(t)) at each time point (t). Analysis was performed using data from the first 10 minutes, as a function of different time lags (T). In the equation above, G̅F̅P̅ is the average of GFP(t) and v̅e̅l̅ is the average of vel(t) over the ten minute interval, while σ_{GFP} and σᵥₑₗ are their standard deviations, respectively. m is the length of the data arrays. This analysis was performed at both the anodal side (i.e., what becomes the cell rear; averaging across positions +120 to +240 of the kymographs) and cathodal side (i.e., what becomes the cell front; averaging across positions −60 to +60 of the kymographs) to look at the onset of the directional response.

To better assess statistical significance, a confidence interval was calculated for the maximum cross-correlation value of randomly permuted data based on the input data. Using a bootstrapping approach (T. Tsai, supra), 2000 bootstrap samples were generated by randomly permuting the values in each of the GFP(t) and vel(t) arrays independently. For each bootstrap sample, the maximum cross-correlation value was calculated, and a 99% confidence interval was estimated by determining the 1 st and 99th percentiles of the distribution of maximum cross-correlation values from the bootstrap samples.

Quantification of v_{E}/D Ratio and Estimation of Charge. The distribution of Galvanin in an electric field can be described by a combination of diffusion and drift processes. Under the experimental conditions, the expected profile of Galvanin on the flat two-dimensional bottom surface near the coverslip was characterized. Under the imaging conditions (800 nm axial resolution), the measured fluorescence intensities will reflect fluorescence predominantly at this surface, and the biological system is treated as a flat two-dimensional surface. Further, in the analysis the Galvanin profile was considered after long exposure to an electric field (≥3 minutes), where the biased Galvanin distribution appears stable. Under this steady-state condition, the effects of drift and diffusion balance. With the electric field oriented along a single axis (taken to be the x axis in the description below), the protein concentration to decay exponentially along this axis under steady state (S.-H. Huang, et al., Anal. Chem. 94, 4531-4537 (2022)). The probability distribution can be described by

$$P ( x , y ) = {Ae}^{\frac{{v}_{E}}{D} x} , \qquad ( 4 )$$

where v_{E} is the electrophoretic drift velocity and D is the diffusion coefficient. Here, A is a normalization constant that is determined by boundary conditions and the amount of Galvanin in the plasma membrane.

The electrophoretic drift velocity v_{E} is dependent on the force F_{E} acting on the charged protein and drag on the particle,

$${v}_{E} = \frac{{F}_{E}}{ζ} , \qquad ( 5 )$$

where ζ is the drag coefficient. The force F_{E} is determined by the Coulombic interaction between the protein with charge q (or z elemental charges e⁻) and the electric field, given by,

$${F}_{E} = qEf ( κ ) = {ze}^{-} Ef ( κ ) . \qquad ( 6 )$$

The factor f(κ) accounts for counterion screening that will result in a reduced effective (i.e., measurable) charge that depends on the Debye screening length κ⁻¹. From the Einstein relationship, the drag coefficient ζ can be related to Galvanin's diffusion coefficient through

$$ζ = \frac{{K}_{B} T}{D} , \qquad ( 7 )$$

where K_{B} is the Boltzmann constant, and T is the temperature. This provides,

$$\frac{{v}_{E}}{D} = \frac{{ze}^{-} Ef ( κ )}{{K}_{B} T} , \qquad ( 8 )$$

which describes an expected relationship between the drift velocity, diffusion coefficient, protein charge, and the strength of the electric field (G. M. Allen, supra).

The counterion screening was using the Debye-Hückel approximation that describes the electric double layer of counter ion screening. This indicates that a tightly bound layer of counter ions move with the charged protein, whose size is larger than the screening length scale and which will result in an effective charge that is less than the bare charge (F. Strubbe, et al., Journal of Colloid and Interface Science 301, 302-309 (2006)). In order to estimate the screening factor f(κ), the approximation that the electric potential ϕ(r) at a distance r from a charge will decay exponentially was used (R. Phillips, et al., Physical Biology of the Cell (ed. 2nd, 2012)),

$$ϕ ( r ) = ϕ \cdot {e}^{- κ r} . \qquad ( 9 )$$

The effective charge will be directly proportional to the electric potential,

$$ϕ ( r ) ∝ q ( r ) ∝ {qe}^{- κ r} , \qquad ( 10 )$$

because of the charge shielding. Taking the screening length to be the Debye length, r=κ⁻¹, an effective charge is given by 0.37ze⁻. The estimated number of elemental charges reported in the main text figures is given by z.

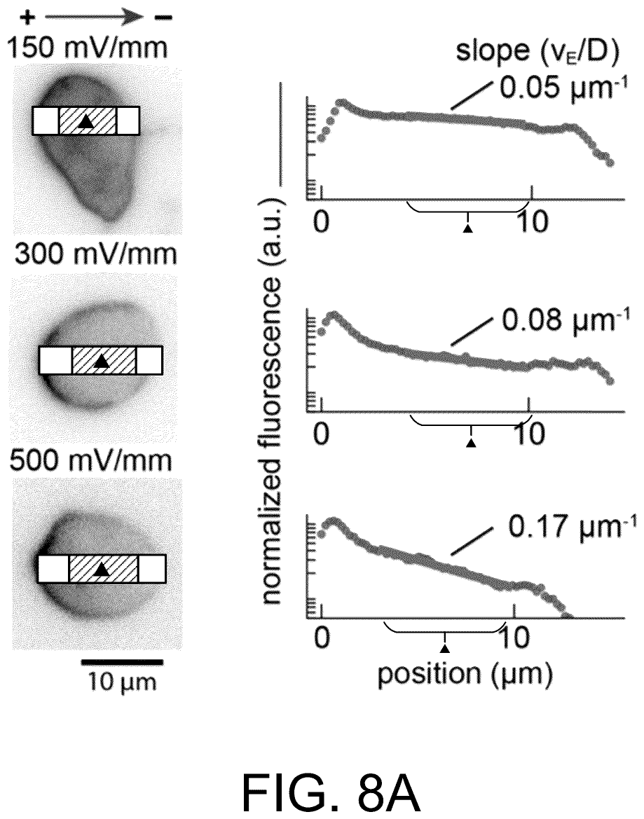

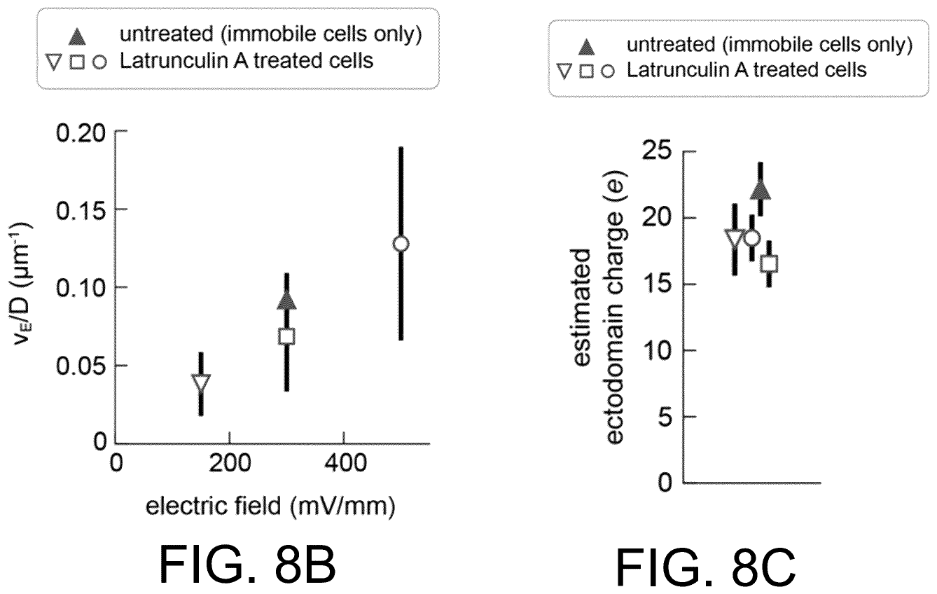

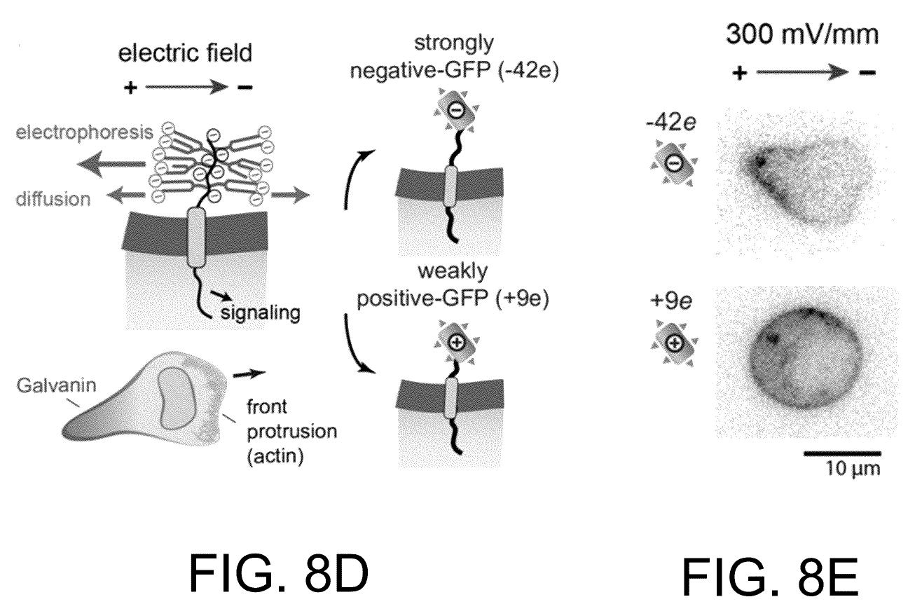

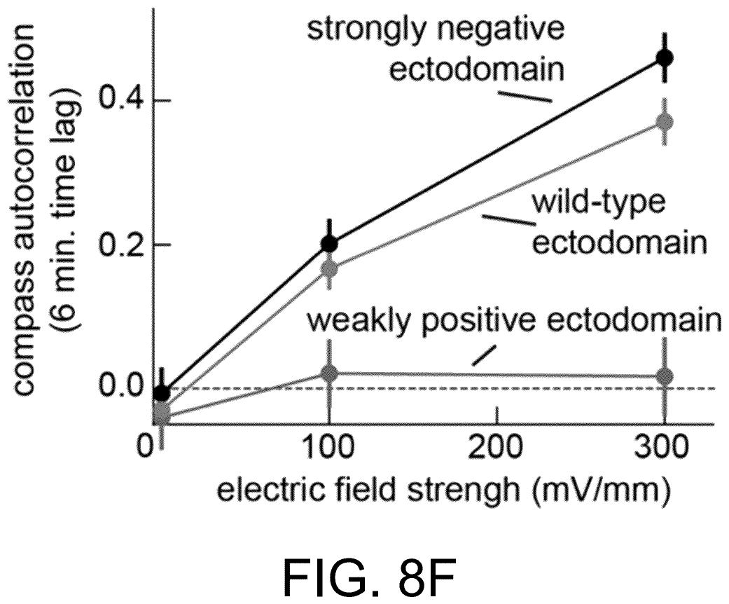

FIGS. 8A-8F illustrate that the sensory mechanism of Galvanin depends on a highly charged ectodomain. FIG. 8A, in the left column, illustrates example micrographs of Galvanin-GFP in individual cells exposed to an electric field for 10 minutes. Cells were treated with Latrunculin A to immobilize them following initial migration under agarose. The intensity profile along the electric field vector direction was quantified to estimate the slope in the decay in fluorescence intensity, expected to be proportional to vE/D. FIG. 8A, in the right column, illustrates semilogarithmic plots of corresponding fluorescence profiles for the examples shown, with a black border indicating the entire cell width, and diagnonal lines or a triangle corresponding to the fit region, selected to avoid the more intense cell periphery. The estimated vE/D values are indicated for each fit. FIG. 8B illustrates a summary of vE/D values across the different electric field strengths. Error bars indicate standard deviation across 13 cells (150 mV/mm), 23 cells (300 mV/mm), and 26 cells (500 mV/mm). Analysis of non-motile cells present in the experiments of FIG. 3 are also included (4 cells). FIG. 8C illustrates estimated ectodomain charge based on the vE/D values of FIG. 8B. Error bars indicate standard error of the mean. FIG. 8D, in the left column, illustrates a schematic illustrating the model of Galvanin electrophoresis due to the net negative charge. Branched changes represent expected glycosylation of ectodomain based on 6 predicted O-/N-type modifications. FIG. 8D, in the right column, illustrates the ectodomain was altered using engineered GFP proteins with either a strongly negative charge (−42e net ectodomain) or weak charge (+9e net ectodomain charge). FIG. 8E illustrates representative fluorescence micrographs of cells expressing the engineered Galvanin constructs exposed to a 300 mV/mm electric field (imaging performed over 2-3 data; −42e, n=40 cells; +9e, n=31 cells). The strongly negative construct shows localization to the anodal side of the cell, similar to the wild-type Galvanin-GFP, while the weakly positive construct remains uniformly distributed. FIG. 8F illustrates compass autocorrelation of cells migrating in a collagen gel. The −/− Galvanin knockout cells expressed Galvanin-GFP, −42GFP-Galvanin, or +9GFP-Galvanin. Autocorrelation analysis on the cosine θ between cell trajectories and the electric field vector. Data points represent average across analysis performed on individual cell tracks (180-750 cells per cell line and per electric field strength); error bars: standard deviation of the mean.

As shown in FIG. 8A, the v_{E}/D ratio was quantified across individual cells. Here a region was selected in the middle of each cell 10-20 pixels thick, adjusted based on the cell shape or presence of bright fluorescence features. Background cellular autofluorescence was first subtracted from the intensity values, determined by measuring the intensity near the cathodal edge of cells exposed to a 500 mV/mm electric field and where no membrane-localized Galvanin-GFP signal was observed. The corrected fluorescence intensities were then log-transformed and the the v_{E}/D ratio was determined from a linear fit of the data using the polyfit( ) function in the NumPy Python package (v. 1.21.6).

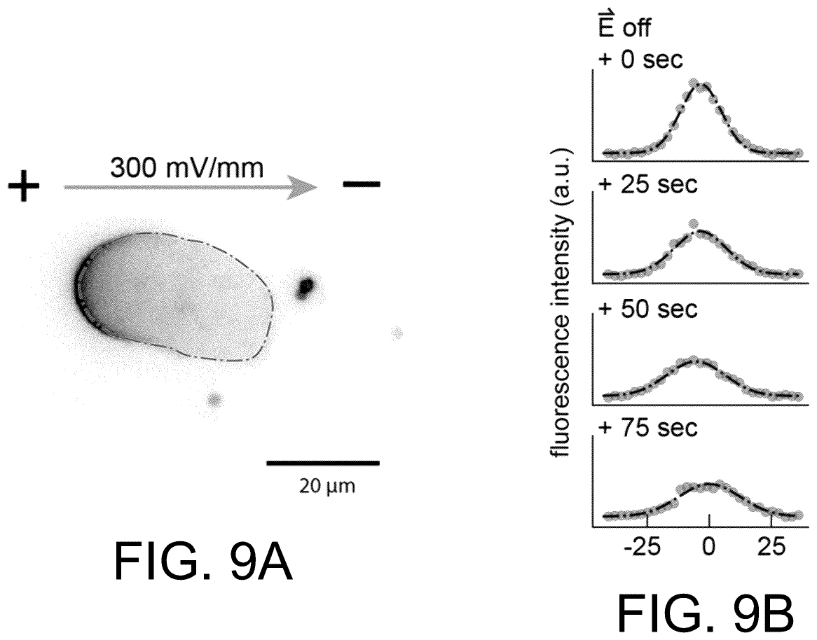

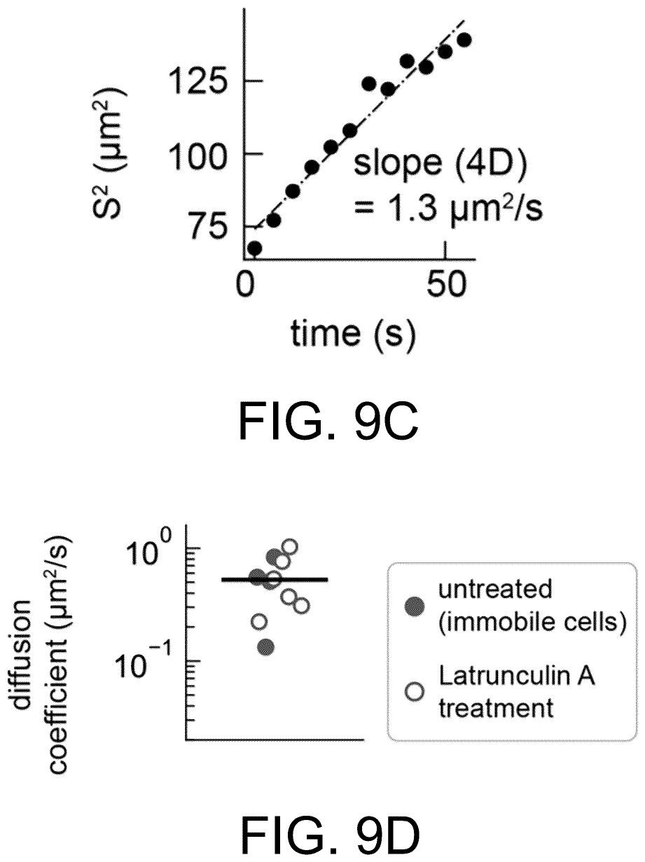

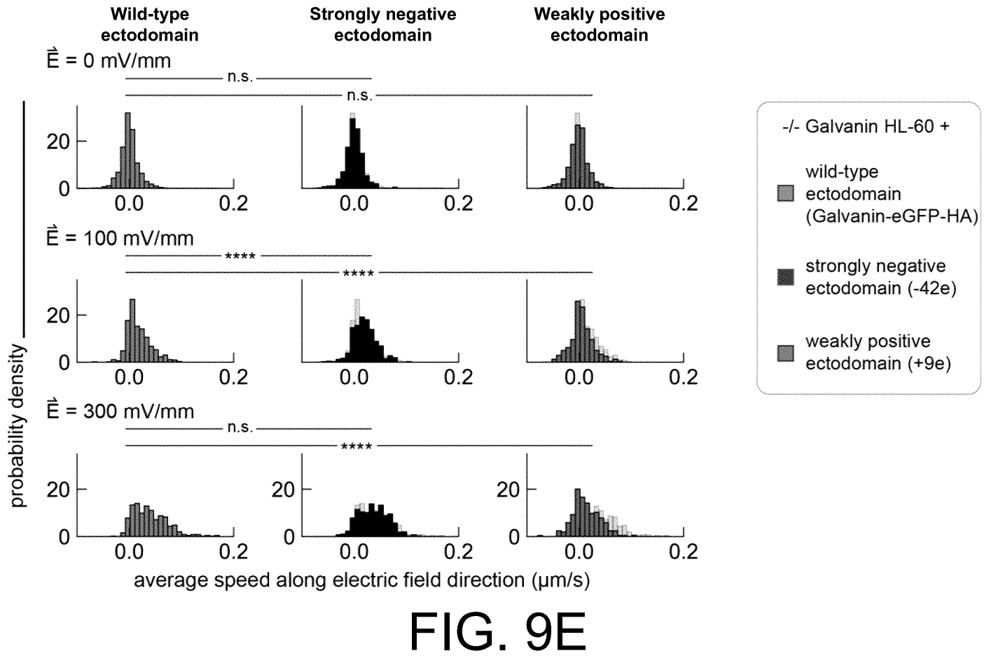

FIG. 9A-9E illustrates that galvanin exhibits rapid membrane diffusion consistent with a single-pass transmembrane protein. FIG. 9A illustrates an example fluorescence micrograph of a cell exposed to an electric field (300 mV/mm) for 5 minutes. The dot-dashed line indicates the cell periphery used in the quantification of Galvanin-GFP fluorescence intensity. FIG. 9B illustrates plots that show the decay in fluorescence intensity at the anodal side of the cell once the electric field is turned off, corresponding to a re-equilibration of Galvanin throughout the plasma membrane. Dot-dash line indicates a Gaussian fit of the fluorescence data. FIG. 9C illustrate the squared width S2, which was used as a measure of the increase in the mean-squared displacement of the fluorescence signal over time, with an expected linear relationship between S2 and 4D, where D is the diffusion coefficient of Galvanin-GFP. FIG. 9D illustrate an estimation of diffusion coefficient. Data points represent measurements from individual cells following the analysis steps of FIGS. 9A-9C. The black line indicates the average across all measurements with an average value of 0.53 μm2/s (±0.1 SEM). FIG. 9E illustrate histograms summarizing average speed along the electric field direction in the Galvanin knockout cell line, expressing Galvanin-GFP, −42GFP-Galvanin, or +9GFP-Galvanin. Galvanin rescue histograms are plotted behind with the −42GFP-Galvanin and +9GFP-Galvanin data for reference. Individual measurements represent the average speed per cell along the electric field direction with 3 minute tracking intervals. Measurements represent 260-700 cells per condition (p-value<0.0001, two-sided Mann-Whitney U test).

Quantification of Galvanin's Diffusion Coefficient. Galvanin quickly returns to a uniform distribution through diffusion in the plasma membrane after removal of the electric field. The fluorescence signal of Galvanin-GFP at the cell periphery (FIG. 9A) represents a one-dimensional slice of this two-dimensional process. This fluorescence is peaked at the anodal side of the cell and can be described by a Gaussian distribution (FIG. 9B). The method used by the PIPE (photo-converted intensity profile expansion) approach (R. Gura Sadovsky, et al., Cell Reports 18, 2795-2806 (2017)), which is to estimate the diffusion coefficient by quantifying the spatial expansion of our fluorescence signal over time, was used. This involves fitting the fluorescence profile to a Gaussian distribution, with the expectation that the squared-width of the Gaussian distribution will increase linearly over time. This is valid at early time frames since the width of the fluorescence peak is already quite wide relative to the length scale of the entire cell and therefore only consider the 1-1.5 minutes following removal of the electric field (FIG. 9C).

To quantify the fluorescence around the periphery, custom code with the Shapely Python package (v. 2.0.4) was used to convert the cell masks into a cell line edge, which was used to generate 300 equally spaced points along the cell boundary. For each point, a line segment orthogonal to the cell edge was generated, and the maximum fluorescence intensity was calculated along that line segment to generate a fluorescence profile around the cell periphery. This intensity profile was fit to a Gaussian function using the curve_fit( ) function in the SciPy Python package (v. 1.9.1). Following the approach of ref. (Gura Sadovsky, supra), the intensity data I(k, t) was fit to a Gaussian function of form,

$$I ( k , t ) = A \cdot {e}^{\frac{- {( k - \overline{k} )}^{2}}{{S}^{2} ( t )}} , \qquad ( 11 )$$

where A is a normalization factor related to the fluorescence intensities, k is the distance along the periphery of the cell, t is the time, k̅ is the mean value where the intensity is peaked, while the variance θ²=S²(t)/2. The diffusion equation follows an identical functional form, and the width of the profile can be related to the diffusion coefficient, where S²(t)=S₀²+4Dt. S₀ is the initial width of the peak profile. The square widths S²(t) from these fits as a function of time were then fitted using the polyfit( ) function in the NumPy Python package (v. 1.21.6) to extract the slope and diffusion coefficient (slope=4D).

### Immunolabeling for Western Blots.

Whole-cell protein lysates were collected from differentiated HL-60 cells for Western blot analysis. For each sample, 5×10⁶ cells were collected, washed once in ice-cold PBS, and resuspended in two volumes of 5× Laemmli SDS-PAGE sample buffer by weight (e.g., 40 μL for 20 mg cell pellet). Samples for heated to 98° C. for 5 minutes and then vortexed briefly prior to sonication with a bath type sonicator (Diagenode of Liege, Belgium; #B01020001). Sonication was performed on their high-power setting at 4° C. with five cycles of 30 seconds on and 30 seconds off. Samples were stored at −20° C. and re-heated to 98° C. prior to gel electrophoresis.

Samples were run on 12% polyacrylamide gels with a protein ladder (Bio-rad of Hercules, CA; #1610317) and transferred to nitrocellulose membranes (Bio-rad, #1620233) by semi-dry transfer in buffer 10 mM 3-(cyclohexylamino)-1-propanesulfonic acid (CAPS) pH 11, 10% methanol. Transferred protein was confirmed and normalized using a reversible total protein stain kit (Pierce of Waltham, MA; #24580). Blots were then blocked in Tris-buffered saline with 0.1% Tween 20 (TBST) with 5% non-fat milk (spun at 2,500 g to remove milk precipitates). Blocking was performed for 30 minutes at room temperature. Protein loading and transfer efficiency was also assessed by staining the residual protein in the gel using Coomassie stain (0.006% Coomassie R250 with 10% acetic acid). Primary antibodies were diluted in TBST with 0.5% non-fat milk and incubated overnight at 4° C. Blots were washed with TBST for 30 minutes, with buffer exchanged every five minutes, and then stained with a horseradish peroxidase (HRP) conjugated secondary antibody diluted in TBST with 0.5% non-fat milk. Following incubation for 60 minutes at room temperature, the blots were washed for 60 minutes in TBST, with buffer exchanged every five minutes. The blots were imaged with a digital gel documentation system (Azure Biosystems of Dublin, CA; c600), allowing for detection of the secondary HRP antibody detected using a chemiluminescence peroxidase substrate kit (MilliporeSigma, #CPS1A120).

Antibodies and Dilution Information. Primary antibody: Polyclonal anti-human TMEM154 (1:500; Proteintech of Rosemont, IL; #24812-1-AP). The polyclonal antibody was found to produce substantial background when blotted against the differentiated HL-60 neutrophil samples. A secondary-only control blot confirmed the background resulted from the primary antibody. To reduce background, prior to probing blots, the diluted primary antibody sample was preabsorbed with total protein from the differentiated HL-60 Galvanin knockout sample bound to nitrocellulose. Briefly, 10 μL of SDS-PAGE sample was diluted with 40 μL water and 2 μL 3 M KCI added to precipitate potassium SDS. The precipitant was removed by centrifugation and the supernatant was diluted in 3 mL TBST and incubated with a piece of nitrocellulose of 50 cm² at room temperature for one hour. Following several washes in TBST, the nitrocellulose was blocked using TBST with 5% non-fat milk for one hour at room temperature. The diluted polyclonal anti-TMEM154 antibody (3 mL total, 1:500 diluted in TBST with 0.5% non-fat milk) was then incubated with this nitrocellulose for one hour for preabsorption prior to use for Western blot.

Secondary antibody: HRP-linked anti-rabbit IgG (1:3000; Cell Signaling Technology of Danvers, MA; #7074S).

### Results

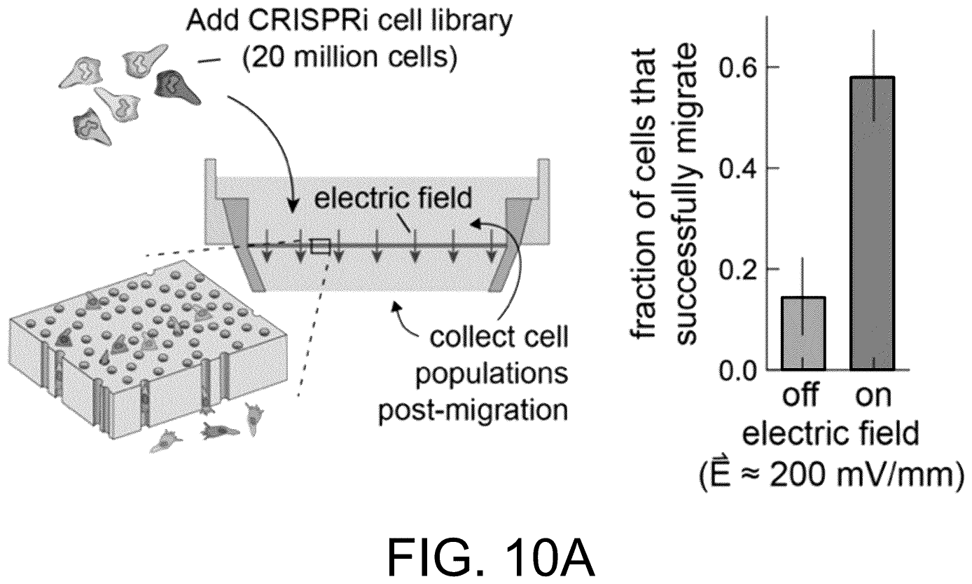

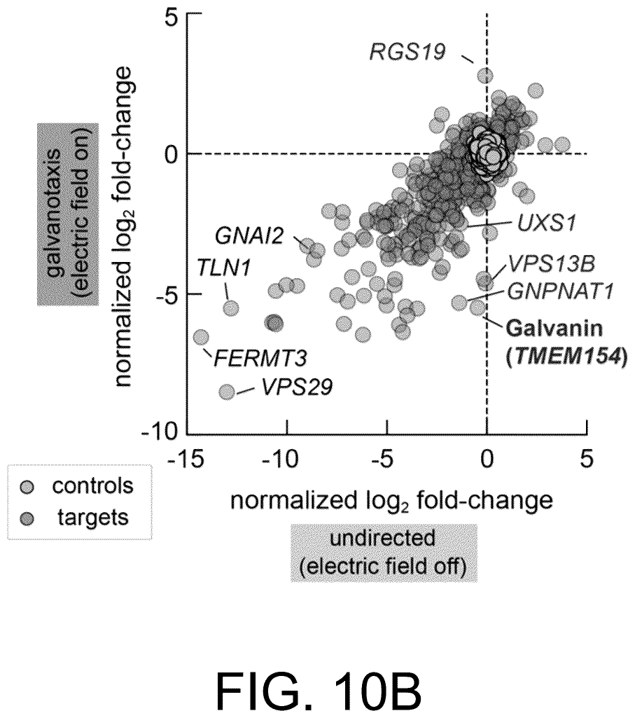

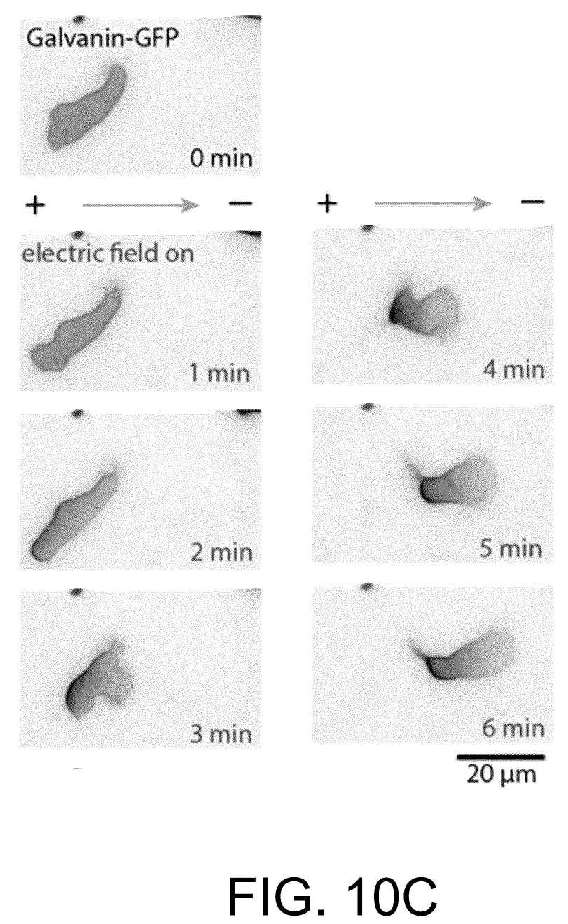

FIGS. 10A-10C illustrate CRISPR interference screen identifies Galvanin as a putative electric field sensor for directed cell migration. FIG. 10A, in the left column, illustrates a schematic of galvanotaxis screen. A pooled CRISPRi library of dHL-60 neutrophil-like cells migrate through a membrane with 3 μm diameter pores. Collection of the sub-population that migrate through and, separately, those that remain above the membrane, allow sgRNA target enrichment and gene candidate identification. FIG. 10A, in the right column, illustrates a fraction of cells collected in the bottom reservoir after two hours. FIG. 10B illustrates a summary of focused library screen targeting 1,070 genes for knockdown. Data points show the normalized log 2 fold-change averaged across three sgRNAs per gene across independent experiments (11 replicates for galvanotaxis and 10 replicates for undirected migration). Control values were generated by randomly selecting groups of three control non-targeting sgRNAs. FIG. 10C illustrates fluorescence micrographs show rapid localization of Galvanin (TMEM154)-GFP toward the anodal pole when differentiated HL-60 neutrophils are exposed to an electric field (300 mV/mm).

Human neutrophils and neutrophil-like cell lines exhibit impressively rapid directional migration in response to a wide variety of chemical cues (Petri and Sanz, supra), and also migrate toward the cathode in an applied electric field (Zhao 2006, supra). The HL-60 cell line, originally derived from a patient with promyelocytic leukemia, can be differentiated in culture to a neutrophil-like cell phenotype (Collins, supra) and is amenable to genome-wide unbiased genetic screen using CRISPR or CRISPR-interference (CRISPRi) approaches (Belliveau, supra; Nagy, supra). This Example describes the development of a device to spatially separate millions of differentiated HL-60 cells based on their capacity to migrate toward the cathode upon exposure to physiologically relevant electric field strengths (FIG. 10A, further detailed in FIGS. 6A-6D, 7A-7E). Cells were added to the top of a membrane containing 3 μm diameter pores and directed downward by applying an electric field with the anodal side above and the cathodal side below the membrane. Both the cells that successfully migrated through the membrane from the bottom reservoir and those that remained above could be collected using the device described herein. Application of an electric field of 200 mV/mm substantially increased the fraction of cells that could be recovered in the bottom reservoir (FIG. 10B), with 60% of the initial cell population recovered after two hours. In contrast, undirected migration in the same device in the absence of an electric field resulted in recovery of only 15% of the initial cell population in the lower reservoir.

A genome-wide unbiased genetic screen was performed in two phases. The initial screen was performed using a genome-wide CRISPRi cell library including sgRNAs targeting 18,901 genes, which was used to narrow the focus in a secondary screen targeting only the 1,070 genes whose knockdown showed the strongest effects on cell migration probability. This approach enabled increased coverage per sgRNA in the secondary screen, thereby improving the statistical confidence for the effects of individual gene candidates (see Supplemental Methods). To better distinguish between perturbations specific to sensing the electric field versus those affecting cell migration more generally, results were compared from two experimental conditions: a) exposure of cells to an electric field (200 mV/mm) and b) undirected cell migration in the same device in the absence of an electric field. In the secondary screen, 473 genes were found where CRISPRi knockdown significantly disrupted migration in the presence of an electric field and 544 genes whose knockdown significantly affected undirected migration, relative to control sgRNAs present in the library (FIG. 10B; adjusted p-value with cutoff of 0.05). The majority of these candidate genes had significant effects in both conditions (Pearson correlation r=0.8 between normalized log₂ fold-change values), reflecting the commonality of migration machinery necessary during neutrophil migration regardless of the nature of the external cue (Belliveau, supra; Pollard and Borisy, supra; T. D. Pollard, et al., Annu Rev Biophys Biomol Struct 29, 545-576 (2000)). A smaller subset of 111 genes, however, were identified as significantly altering cell migration probability only in the presence of the electric field. This subset included TMEM154 (transmembrane protein 154), that are referred to as Galvanin, which stood out as the transmembrane protein whose knockdown gave the strongest electric-field-specific phenotype.

Galvanin is predicted to be a single-pass transmembrane protein (161 amino acids, UniProt ID: Q6P9G4). In primary neutrophils, its transcriptional levels are roughly similar to other integral membrane proteins such as the LPS receptor CD14 and the integrin α_{M} (E. Rincón, et al., BMC Genomics 19, 573 (2018)). It was confirmed that Galvanin localizes to the HL-60 cell plasma membrane by tagging the intracellular C-terminus with eGFP. As a putative electric field-sensitive protein, its localization was indicated to become spatially biased when cells were exposed to an electric field. Using an agarose overlay to confine migrating cells to a single plane on a glass coverslip, the cells were exposed to an electric field of 300 mV/mm. Relocalization of Galvanin-GFP was observed, which rapidly becomes biased to the anodal (positive) pole of the cell within one minute of exposure (FIG. 10C). For these cells, which migrate toward the cathode, this equates to relocalization of the protein to the cell rear and is consistent with electrophoresis of a net negatively charged protein. From the amino acid sequence alone, the ectodomain is indicated to have a net charge of −7e, which is too low to account for such a notably biased distribution (G. M. Allen, supra). One likely explanation for this disparity is glycosylation of the ectodomain, which could substantially increase its negative charge. Protein glycosylation, referring to the addition of carbohydrates to the polypeptide following translation, has been implicated in galvanotaxis of other cell types (Belliveau, supra; Nagy, supra; Lawrence, supra). These modifications often form branched oligosaccharide chains that terminate in negatively charged sialic acid groups and can confer a strongly negative net charge on glycosylated proteins at neutral pH (Bodor, supra). Using NetNGlyc 1.0 (Sarkar, supra) and NetOGlyc 4.0 (Y.-J. Huang, supra), six amino acid positions in the ectodomain of Galvanin were found that may contain glycosylation (one N-type and up to five O-type modifications).

Several of the screen candidate genes were followed up on to better assess how the genetic perturbations altered cell migration. In addition to Galvanin itself, three other candidate genes were focused on that might be expected to affect the cell surface presentation of a putative electric field sensor: VPS13B which is involved in membrane trafficking, and GNPNAT1 and UXS1 which are both enzymes involved in protein glycosylation. Individual stable HL-60 knockdown lines were generated for each of these four candidates, then differentiated them and embedded them in three-dimensional collagen gels, where their speeds and migration directions were measured using video microscopy. Consistent with the screen results, each cell line appeared to migrate normally in the absence of an electric field. In the presence of an electric field, each cell line showed reduced directionality when compared to migration of cells with a non-targeting sgRNA (FIG. 7E), indicating their involvement in the directional response.

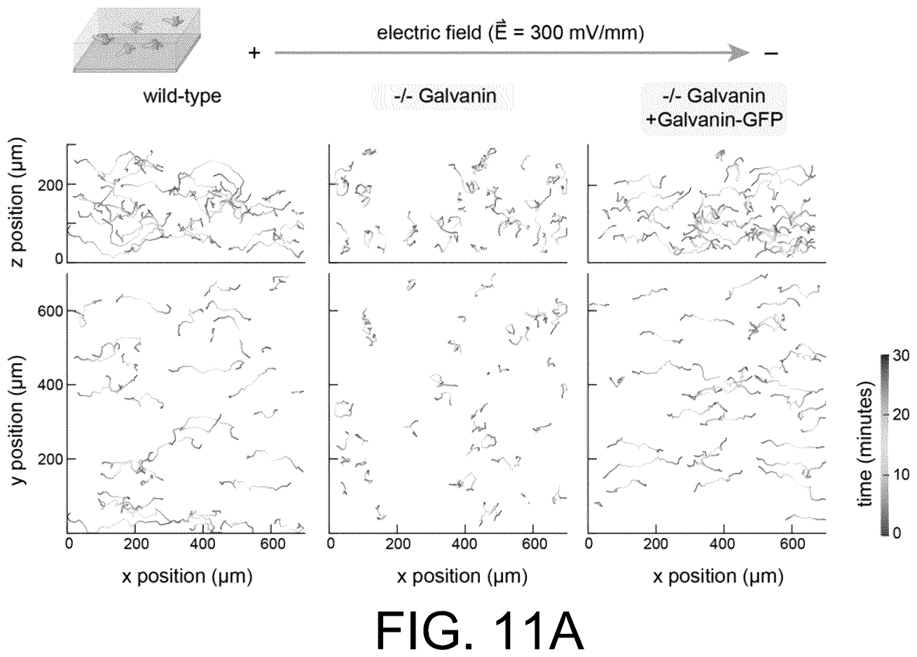

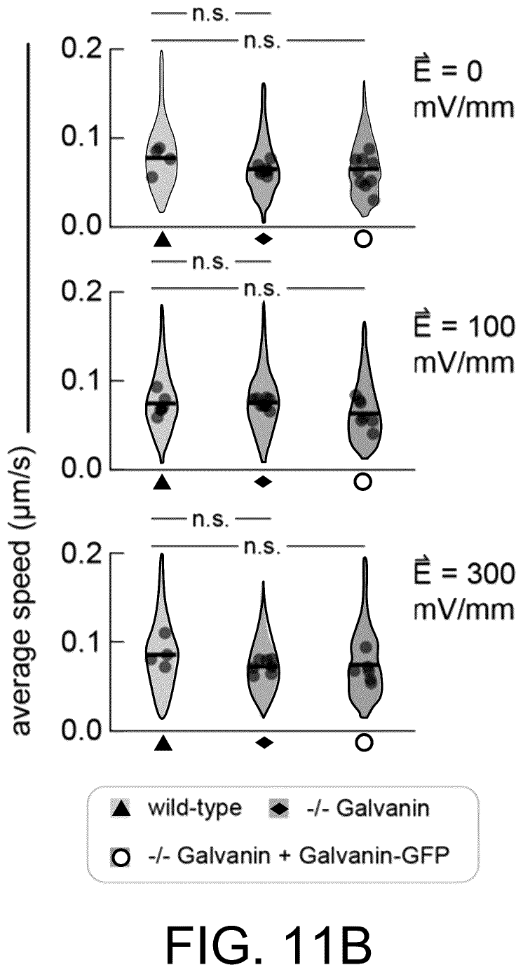

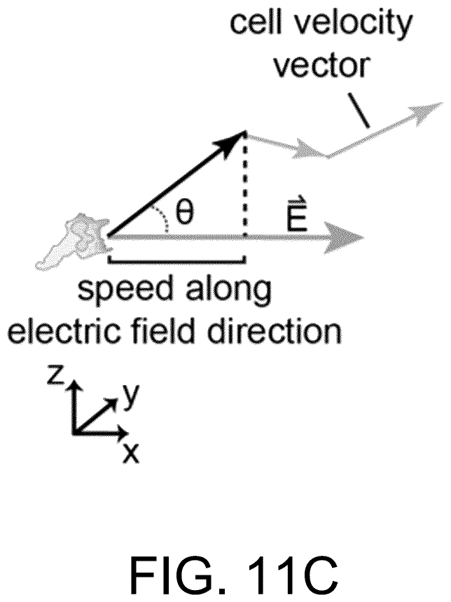

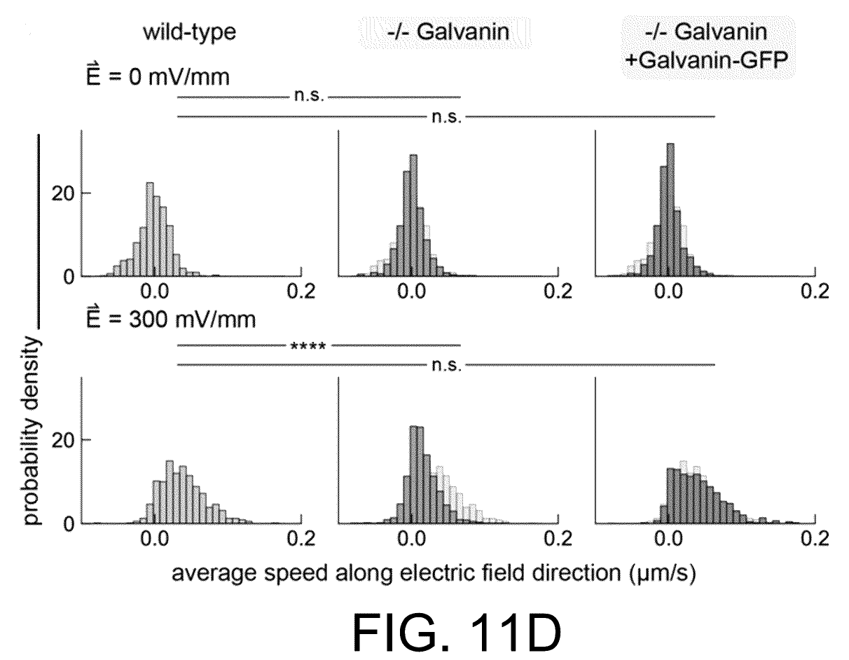

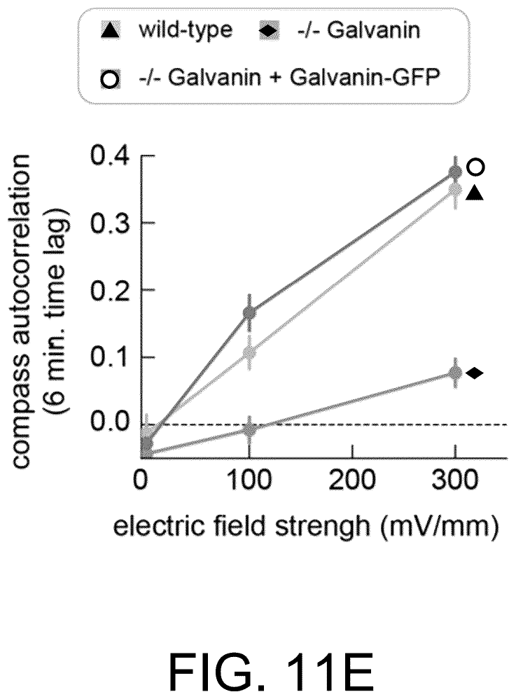

FIGS. 11A-11E illustrates that galvanin is critical for persistent cathodal migration in HL-60 neutrophils. FIG. 11A illustrates that nuclear tracking was performed during migration of HL-60 neutrophils in a collagen gel exposed to an electric field (300 mV/mm). Data represents a subset of data, with only 50 cell tracks shown per cell line (dHL-60 neutrophil wild-type, -/-Galvanin knockout, and the genetic rescue with Galvanin-GFP). FIG. 11B illustrates average speed calculated based on nuclear tracking with 3 minute time intervals. Individual data points represent average values across cells from a single field of view. The shaded regions show a kernel density estimation of the distribution of measurements across all fields of view (two-sided Mann-Whitney U test found no significant difference). FIG. 11C illustrates a schematic showing calculation of directed speed along the electric field vector. FIG. 11D illustrates histograms summarize average speed along the electric field direction. Individual measurements represent the average speed per cell along electric field direction, with 3 minute tracking intervals (400-1,000 cells and 4,000-9,500 tracking intervals per condition). Wild-type histograms (light gray) are plotted along with the −/− Galvanin knockout and genetic rescue data for reference (p-value<0.0001, two-sided Mann-Whitney U test). FIG. 11E illustrates that longer-term directed movement was determined by defining a compass autocorrelation. For individual cell tracks, the angle θ between the cell vector and the electric field vector was determined for each 3 minute time interval. Autocorrelation analysis was performed on the set of corresponding cosine θ values and averaged across cells. Autocorrelation values for a six minute time lag are shown here, with all time lags shown in FIGS. 7A-1E. Error bars represent standard error of the mean. For each cell type in FIGS. 11B, 11D, and 11E, 400-1,000 cells were analyzed per condition (30-150 cells quantified per imaging acquisition, with 4-9 acquisitions per cell line and per electric field strength).

To better assess the functional contribution of Galvanin to directional migration, CRISPR-Cas9 was used to create two clonal knockout cell lines where the endogenous Galvanin gene was disrupted (FIG. 4A). Their directional migration was then assessed in collagen gels when exposed to electric fields of varying strength. In FIG. 11A, tracks of individual migrating cells exposed to a 300 mV/mm field are shown, tracking cell nuclei over a 30-minute period. A notable loss of directed migration was observed in each Galvanin knockout cell line relative to wild-type cells (FIG. 11A, middle, FIG. 4B). By expressing the Galvanin-GFP construct in the Galvanin knockout cell line, the cathodal directional migration of wild-type cells was rescued (FIG. 11A, right), demonstrating that Galvanin is necessary for the normal electric field response. Notably, the Galvanin knockout cells were still highly migratory, with average migration speeds indistinguishable from wild-type cells in the presence or absence of an electric field (FIG. 11B); that is, their phenotypic defect lies strictly in their ability to orient toward the cathode and not in any other aspect of cell migration. The average speed of individual cells projected along the electric field vector (that is, the electric field-directed component of the cell speed) was quantified (FIG. 11C). Plotting the average speeds across all cells tracked, a significant loss of directionality was found in the knockout cell line compared to the wild-type and Galvanin rescue cell lines (FIG. 11D).

The directional movement at longer length scales was assessed. For migration in a physically complex environment such as fibrous collagen gel, which results in somewhat tortuous cell trajectories, a metric was chosen to assess the overall directional bias under a sustained cue. Specifically, the autocorrelation of measured cosine of the angles (cos 0) between the migration vector of individual cells and the electric field vector was quantified, which is referred to as the compass autocorrelation (FIG. 4C). This metric remains high when cells move over long periods of time in the same net direction, even if the local path persistence is relatively low (as is typical for rapidly moving neutrophils). Cells exhibited a compass autocorrelation that was dependent on the strength of the electric field and was notably reduced with the Galvanin knockdown cell lines (FIG. 4D). In FIG. 11E the compass autocorrelation was plotted at a six-minute time lag, with the Galvanin knockout cell line showing a substantial loss of directional movement along the electric field vector, and complete rescue by Galvanin-GFP. These results indicate that Galvanin affects directed migration during galvanotaxis of these rapidly moving neutrophil-like cells.

To better understand how Galvanin supports directed migration, the dynamics of its relocalization on the membrane was measured while also measuring changes in local protrusion and retraction activity and front-rear cell polarization (Bodor, supra; Pincus and Theriot, Journal of Microscopy 227, 140-156 (2007)). Using the under-agarose assay to confine cells to migrate in a single plane, cell migration was monitored at high spatial and temporal resolution during five minutes of undirected migration, five minutes of exposure to an electrical stimulus of 300 mV/mm, and five minutes of recovery (FIG. 5A). Galvanin-GFP localization was measured around the periphery of individual cells, and cell migration changes were quantified using the Adapt package (Barry, supra), which measures the relative local protrusion or retraction of the cell edge, as a function of position around the cell perimeter. This analysis was performed on 46 cells, enabling generation of population-averaged kymographs of Galvanin localization dynamics (FIG. 5B) and an associated mapping of protrusion and retraction (FIG. 5C). It was found that the cellular response to the electric field is almost immediate in this Example, with a relocalization of Galvanin-GFP to the anode that was nearly complete within one minute (FIG. 5D). Accompanying this was a notable change in the spatial distribution of protrusion and retraction, with an immediate increase in retraction at the newly forming cell rear and an increase in protrusion at what becomes the cell front (FIG. 5E). To compare the timing of the Galvanin-GFP relocalization with the changes in protrusion and retraction activity, a cross-correlation was performed to compare these two parameters over time (FIG. 5F). On both the rear (anodal) and front (cathodal) sides of the cell, the Galvanin-GFP relocalization was tightly coupled to the protrusion/retraction activity, with a maximum negative correlation at zero time lag, indicating that the two processes are nearly simultaneous (within the time resolution of these experiments). This finding was also maintained when measuring the cross-correlation between Galvanin-GFP signal and protrusion/retraction activity for individual cells (FIG. 5G). These results indicate that Galvanin relocalization defines the cell front and cell rear for directed cell migration during exposure to an electrical cue, either by locally activating retraction at the rear, or by removing inhibition of protrusion at the front.

In theory, the rapid anode-directed electrophoresis of Galvanin should depend on the Coulombic interaction between the charged ectodomain of the protein and the electric field, with the field unable to penetrate into the intracellular space (M. M. Klee, American Journal of Physics 82, 451-459 (2014); Poo and Robinson, Nature 265, 602-605 (1977); T. Taghian, et al., Journal of The Royal Society Interface 12, 20150153 (2015)). The stable spatial distribution of Galvanin-GFP observed after several minutes of electric field exposure represents a balance between the Coulombic movement toward the positive, anodal pole with electrophoretic velocity v_{E} and equilibration by diffusion with an effective diffusion coefficient D (G. M. Allen, supra). Specifically, the steady-state Galvanin concentration will vary along the electric field vector with an approximate exponential decay (S.-H. Huang, et al., Anal. Chem. 94, 4531-4537 (2022)), with a characteristic length that depends on the ratio v_{E}/D. This ratio can be used to infer the net charge on the protein. To more precisely quantify this ratio and avoid confounding factors introduced by migrating cells (including possible membrane flow), the under-agarose experiments were repeated in the presence of Latrunculin A. This drug inhibits filamentous actin assembly and allows immobilization of cells as they begin to migrate under the agarose overlay, maintaining their flattened shape against the coverslip. Latrunculin A-treated cells were exposed to electric field strengths of 150 mV/mm, 300 mV/mm, and 500 mV/mm (FIG. 8A). The slopes of the fluorescence intensity profiles, plotted on a semilogarithmic scale, were then used to estimate the ratio v_{E}/D at each electric field strength. As shown in FIG. 8B, the v_{E}/D ratio increased from 0.05 μm⁻¹ to 0.2 μm⁻¹ over this range. The net charge is proportional to the v_{E}/D ratio, normalized by the electric field strength, and a similar estimate of the net charge was determined on Galvanin across all conditions, with a value of −18e (±1.1 SEM) averaged across all the data (FIG. 8C). Using this same dataset, the effective diffusion coefficient D was estimated for Galvanin-GFP by quantifying its equilibration back to a uniform distribution after the electric field stimulus was removed (FIGS. 9A-9D), obtaining a value for D of 0.53 μm²/s (±0.1 SEM), which is consistent with expectations for a single-pass transmembrane protein (R. Worch, et al., J Membr Biol 250, 393-406 (2017)). In summary, its high mobility and high net negative charge enable Galvanin to exhibit rapid dynamics as an electric field sensor.

Due to the limited structural characterization of Galvanin, it remained unclear whether additional features of the protein might be important for its sensory function, or whether the net charge of the ectodomain was by itself sufficient. To test this, the wild-type ectodomain was removed and replaced with engineered protein domains that have a known net negative charge (FIG. 8D). Previously developed “super-charged” GFP proteins were utilized (Lawrence, supra). A highly negative construct was expressed, replacing Galvanin's ectodomain with a negatively charged XTEN linker domain (V. Schellenberger, et al., Nat Biotechnol 27, 1186-1190 (2009)) and negatively charged version of GFP, yielding an expected total net charge of −42e at pH 7. Additionally, a construct was expressed in which the ectodomain was replaced with a weakly positive GFP, having an expected net charge of +9e at pH 7. Although these mutated forms of GFP exhibited poorer fluorescence, with the +9e form showing more variable membrane and cytosolic fluorescence, both constructs still localized to the plasma membrane in Galvanin knockout cells (FIG. 8E).

The functional activity of these engineered constructs was assessed by expressing them in Galvanin knockout cells and exposing the cells to an electric field. Using Latrunculin A as noted above to immobilize cells, the −42e construct showed a strong anodal localization (FIG. 8E). Estimating v_{E}/D as described above, a net charge was calculated to be 37e (±2.5 SEM), which is close to the calculated value and provides confidence in the charge measurement technique. In contrast, the +9e construct showed no notable change in GFP distribution when cells were exposed to an electric field, indicating that the weaker charge is insufficient for relocalization at these electric field strengths, as had been previously predicted based on theoretical considerations (G. M. Allen, supra). To assess migration in cell lines expressing these engineered constructs, cell tracking assay was implemented in collagen. The −42e construct was able to completely rescue directed cell migration during exposure to an electric field, with even a slightly higher compass autocorrelation than wild-type cells (FIG. 8F). At 100 mV/mm, the −42e construct also led to an increased average speed along the electric field direction when compared to the Galvanin-GFP rescue cell line (FIG. 9E). In contrast, and consistent with the lack of spatial relocalization of the +9e construct, the weakly charged ectodomain was unable to rescue the directed migration response. Accordingly, it was determined that the high net negative charge of the ectodomain is sufficient to mediate the sensory response of Galvanin.

### Example Clauses

1. A device, including: a first reservoir containing cell media; a second reservoir containing the cell media; a porous membrane disposed between the first reservoir and the second reservoir, the porous membrane including pores that are permeable to cells; and a stimulus generator configured to induce a differential stimulus between the first reservoir and the second reservoir across the porous membrane, the differential stimulus causing migration of at least a portion of the cells between the first reservoir and the second reservoir across the porous membrane.

2. The device of clause 1, wherein the porous membrane has a thickness in a range of about 10 microns to about 100 microns, and/or wherein the pores have widths in a range of about 1 micron to about 15 microns.

3. The device of clause 1 or 2, wherein the stimulus generator includes: a first electrode electrically connected to the first reservoir; a second electrode electrically connected to the second reservoir, and a power source configured to induce a voltage across the first electrode and the second electrode.

4. The device of clause 3, further including: a first agarose salt bridge disposed between the first electrode and the first reservoir; and a second agarose salt bridge disposed between the second electrode and the second reservoir.

5. The device of any of clauses 1-4, wherein the stimulus generator includes: a pump configured to output a chemical in the second reservoir, a concentration of chemical in the first reservoir being different than a concentration of the chemical in the second reservoir.

6. The device of any of clauses 1-5, wherein the stimulus generator includes: an electromagnet configured to induce a magnetic field.

7. The device of any of clauses 1-6, wherein the differential stimulus includes at least one of an electrical signal, a chemical signal, or a magnetic signal.

8. The device of any of clauses 1-7, further including: a housing at least partially enclosing the first reservoir, the second reservoir, and the porous membrane.

9. The device of any of clauses 1-8, the porous membrane being a first porous membrane, the pores being first pores, the stimulus generator being a first stimulus generator, the differential stimulus being a first differential stimulus, the portion of the cells including a first portion of the cells, the device further including: a third reservoir including the cell media; a second porous membrane disposed between the second reservoir and the third reservoir, the second porous membrane including second pores that are permeable to the cells; and a second stimulus generator configured to induce a second differential stimulus between the first reservoir and the second reservoir across the porous membrane, the second differential stimulus causing migration of at least a second portion of the cells between the second reservoir and the third reservoir across the second porous membrane, wherein the first differential stimulus is different than the second differential stimulus.

10. A device, including: a substrate; a first layer disposed on the substrate, cells being disposed between the substrate and at least a portion of the first layer; a second layer disposed on the first layer, the first layer being disposed between the substrate and the second layer; and a stimulus generator configured to output a stimulus that causes at least a portion of the cells to migrate from the first layer to the second layer.

11. The device of clause 10, wherein the first layer and/or the second layer includes at least one of: collagen, fibrin, elastin, laminin, entactin, alginate, proteoglycans, glycoproteins, epithelial cells, endothelial cells, muscle cells, mucous cells, fibroblasts, or adipocytes, and/or wherein the stimulus includes an electrical signal, a chemical signal, or a magnetic signal.

12. The device of clause 10 or 11, further including: Arginine-Glycine-AsparticAcid (RGD) proteins disposed in the first layer and/or the second layer.

13. A method of collecting cells that migrate in response to a stimulus, the method including: receiving, in a reservoir, a sample including first cells and second cells, the first cells and the second cells having different responses to the stimulus; causing the first cells to migrate through a cell separator by exposing the first cells and the second cells to the stimulus; and outputting a volume including the first cells.

14. The method of clause 13, wherein the stimulus includes at least one of an electrical signal, a chemical signal, or a magnetic signal.

15. The method of clause 13 or 14, wherein causing the first cells to migrate through the cell separator by exposing the first cells and the second cells to the stimulus includes: causing the first cells to migrate into and/or through at least one of: one or more protein layers, one or more tissue layers, one or more collagen layers, one or more fibrin layers, or one or more porous membranes.

16. The method of clause 15, wherein outputting the volume including the first cells includes dissolving the one or more fibrin layers using nattokinase.

17. The method of any of clauses 13-16, the reservoir being a first reservoir, wherein causing the first cells to migrate through the cell separator by exposing the first cells and the second cells to the stimulus includes: causing the first cells to migrate into a second reservoir, the cell separator being disposed between the first reservoir and the second reservoir.

18. The method of any of clauses 13-17, wherein the first cells express a gene and the second cells do not express the gene, or wherein the first cells express a first version of the gene and the second cells express a second version of the gene.

19. The method of any of clauses 13-18, wherein the stimulus includes at least a portion of a candidate therapeutic agent, the method further including: estimating an efficacy of the candidate therapeutic agent by analyzing the volume including the first cells.

20. The method of any of clauses 13-19, wherein the first cells and the second cells are obtained from a subject, the method further including: identifying a pathology of the subject by analyzing the volume including the first cells, wherein the pathology includes at least one of: a cancer type, a cancer subtype, an autoimmune disease, an autoimmune disease subtype, an inflammatory disease, an inflammatory disease subtype, a fibrotic disease, or a fibrotic disease subtype of the first cells.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, the practice of the present disclosure can employ conventional techniques of immunology, molecular biology, microbiology, cell biology and recombinant DNA. These methods are described in the following publications. See, e.g., Green and Sambrook, Molecular Cloning: A Laboratory Manual, 4nd Edition (2012); F. M. Ausubel, et al. eds., Current Protocols in Molecular Biology, (2003); the series Methods In Enzymology (Academic Press, Inc.); Behlke, et al., Polymerase Chain Reaction: Theory and Technology (2019); Greenfield, ed. Antibodies, A Laboratory Manual, Second Edition (2014); and Capes-Davis and R. I. Freshney, eds. Freshney's Culture of Animal Cells 8th Edition (2021).

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

1. A device, comprising:
a first reservoir containing cell media;
a second reservoir containing the cell media;
a porous membrane disposed between the first reservoir and the second reservoir, the porous membrane comprising pores that are permeable to cells; and
a stimulus generator configured to induce a differential stimulus between the first reservoir and the second reservoir across the porous membrane, the differential stimulus causing migration of at least a portion of the cells between the first reservoir and the second reservoir across the porous membrane.

2. The device of claim 1, wherein the porous membrane has a thickness in a range of about 10 microns to about 100 microns, and/or
wherein the pores have widths in a range of about 1 micron to about 15 microns.

3. The device of claim 1, wherein the stimulus generator comprises:
a first electrode electrically connected to the first reservoir;
a second electrode electrically connected to the second reservoir, and
a power source configured to induce a voltage across the first electrode and the second electrode.

4. The device of claim 3, further comprising:
a first agarose salt bridge disposed between the first electrode and the first reservoir; and
a second agarose salt bridge disposed between the second electrode and the second reservoir.

5. The device of claim 1, wherein the stimulus generator comprises:
a pump configured to output a chemical in the second reservoir, a concentration of chemical in the first reservoir being different than a concentration of the chemical in the second reservoir.

6. The device of claim 1, wherein the stimulus generator comprises:
an electromagnet configured to induce a magnetic field.

7. The device of claim 1, wherein the differential stimulus comprises at least one of an electrical signal, a chemical signal, or a magnetic signal.

8. The device of claim 1, further comprising:
a housing at least partially enclosing the first reservoir, the second reservoir, and the porous membrane.

9. The device of claim 1, the porous membrane being a first porous membrane, the pores being first pores, the stimulus generator being a first stimulus generator, the differential stimulus being a first differential stimulus, the portion of the cells comprising a first portion of the cells, the device further comprising:
a third reservoir comprising the cell media;
a second porous membrane disposed between the second reservoir and the third reservoir, the second porous membrane comprising second pores that are permeable to the cells; and
a second stimulus generator configured to induce a second differential stimulus between the first reservoir and the second reservoir across the porous membrane, the second differential stimulus causing migration of at least a second portion of the cells between the second reservoir and the third reservoir across the second porous membrane,
wherein the first differential stimulus is different than the second differential stimulus.

10. A device, comprising:
a substrate;
a first layer disposed on the substrate, cells being disposed between the substrate and at least a portion of the first layer;
a second layer disposed on the first layer, the first layer being disposed between the substrate and the second layer; and
a stimulus generator configured to output a stimulus that causes at least a portion of the cells to migrate from the first layer to the second layer.

11. The device of claim 10, wherein the first layer and/or the second layer comprises at least one of: collagen, fibrin, elastin, laminin, entactin, alginate, proteoglycans, glycoproteins, epithelial cells, endothelial cells, muscle cells, mucous cells, fibroblasts, or adipocytes, and/or
wherein the stimulus comprises an electrical signal, a chemical signal, or a magnetic signal.

12. The device of claim 10, further comprising:
Arginine-Glycine-Aspartic Acid (RGD) proteins disposed in the first layer and/or the second layer.

13. A method of collecting cells that migrate in response to a stimulus, the method comprising:
receiving, in a reservoir, a sample comprising first cells and second cells, the first cells and the second cells having different responses to the stimulus;
causing the first cells to migrate through a cell separator by exposing the first cells and the second cells to the stimulus; and
outputting a volume comprising the first cells.

14. The method of claim 13, wherein the stimulus comprises at least one of an electrical signal, a chemical signal, or a magnetic signal.

15. The method of claim 13, wherein causing the first cells to migrate through the cell separator by exposing the first cells and the second cells to the stimulus comprises:
causing the first cells to migrate into and/or through at least one of:
one or more protein layers,
one or more tissue layers,
one or more collagen layers,
one or more fibrin layers, or
one or more porous membranes.

16. The method of claim 15, wherein outputting the volume comprising the first cells comprises dissolving the one or more fibrin layers using nattokinase.

17. The method of claim 13, the reservoir being a first reservoir, wherein causing the first cells to migrate through the cell separator by exposing the first cells and the second cells to the stimulus comprises:
causing the first cells to migrate into a second reservoir, the cell separator being disposed between the first reservoir and the second reservoir.

18. The method of claim 13, wherein the first cells express a gene and the second cells do not express the gene, or
wherein the first cells express a first version of the gene and the second cells express a second version of the gene.

19. The method of claim 13, wherein the stimulus comprises at least a portion of a candidate therapeutic agent, the method further comprising:
estimating an efficacy of the candidate therapeutic agent by analyzing the volume comprising the first cells.

20. The method of claim 13, wherein the first cells and the second cells are obtained from a subject, the method further comprising:
identifying a pathology of the subject by analyzing the volume comprising the first cells,
wherein the pathology comprises at least one of: a cancer type, a cancer subtype, an autoimmune disease, an autoimmune disease subtype, an inflammatory disease, an inflammatory disease subtype, a fibrotic disease, or a fibrotic disease subtype of the first cells.
